(19) United States
(12) Patent Application Publication
(10) Pub. No.: US 2026/0138989 A1
(43) Pub. Date: May 21, 2026

(54) PATIDEGIB AND PROCESS FOR THE PREPARATION THEREOF

(71) Applicant: Sol-Gel Technologies Ltd., Ness Ziona (IL)

(72) Inventors: Moshe ARKIN, Kfar Shmaryahu (IL); Daniel GENOV, Ness Ziona (IL); Ofer TOLEDANO, Kfar Saba (IL)

(73) Assignee: Sol-Gel Technologies Ltd., Ness Ziona (IL)

(21) Appl. No.: 19/489,997

(22) PCT Filed: Jul. 24, 2025
(86) PCT No.: PCT/IL2025/050641

§ 371 Date: Dec. 4, 2025

Related U.S. Application Data
(60) Provisional application No. 63/675,306, filed on Jul. 25, 2024.

(51) Int. Cl. C07D 491/048; A61K 31/4355

(52) U.S. Cl. CPC C07D 491/048; A61K 31/4355

(57) ABSTRACT

The present invention relates to a compound of patidegib, a process for the preparation thereof, and methods of treatment of BCC and/or preventing, delaying and/or inhibiting the formation of new basal cell carcinoma in a subject in need, by administering the compound of this invention.

### FIELD OF THE INVENTION

The present invention, in some embodiments thereof, relates to a compound of patidegib or any pharmaceutically acceptable salt thereof, wherein the compound comprises less than 0.05 wt % of impurity A or any salt thereof, or less than 0.5 wt % of impurity B or any salt thereof, or any combination thereof. In another aspect, the present invention relates to a process of preparation of said compound, pharmaceutical compositions and methods thereof.

### BACKGROUND

Basal cell carcinoma (BCC) is a common form of skin cancer, a subtype of Non Melanoma Skin Cancer (NMSC). BCC arises from abnormal, uncontrolled growth of basal cells, and is driven by the hedgehog (HH) signaling pathway which is considered to be a major signal transduction pathway during embryonic development, but it usually shuts down after birth. Activated Hedgehog (HH) signaling driven by mutations in the tumor-suppressor gene Patched (PTCH) and/or the G-protein-coupled receptor Smoothened (SMO) is known to promote oncogenic signaling and drives the growth of BCC. Mutations of the human patched gene such as PTCH1 and PTCH2 are associated with nevoid basal cell carcinoma syndrome and basal cell carcinoma.[1]

A variety of surgical and non-surgical therapies are available for BCCs. Nonsurgical therapies include radiation therapy, chemotherapy, and immunotherapy. These therapies can be useful for definitive treatment of primary tumors and some recurrent BCC tumors and for relieving symptoms associated with inoperable tumors. However, some of these therapies also can have significant unpleasant side effects. Side effects of radiation therapy and certain chemotherapies are well documented. One form of immunotherapy involves intralesional injections of interferon. While interferon therapy can be effective against BCC, the multiple intralesional injections can require several clinic visits per week for many weeks and are painful.

The most common topical treatment for BCC is 5-fluorouracil (5FU) and/or imiquimod, which are highly effective but causes a painful erosion in the treatment area.

There are genetic disorders which are associated with an increased risk of developing BCC at an early age and with increased morbidity. One of these disorders is nevoid basal cell carcinoma syndrome—Nevoid basal cell carcinoma syndrome (NBCCS), also known as basal cell nevus syndrome or Gorlin syndrome, is a rare multisystem disorder of autosomal dominant inheritance caused in most cases, not all, by germline mutations of the human patched gene-1 (PTCH1) and more rarely by mutation in SMO, SUFU (SUFU negative regulator of hedgehog signaling) and/or PTCH2 [1,2a, 2b]. Affected patients have both developmental anomalies and postnatal tumors, including multiple BCCs, odontogenic keratocysts, and medulloblastoma [3]. Gorlin syndrome affects individuals which develop multiple (dozens to thousands) of microscopic and macroscopic BCCs, various benign hair follicle hamartomas, palmar, and plantar pits in addition to skeletal defects (bifid ribs and syndactyly), central nervous system abnormalities (calcification of the falx cerebri and agenesis of the corpus callosum), craniofacial features (enlarged skull, hypertelorism, and frontal bossing), and benign odontogenic keratocysts of the jaw.

Additional examples of genetic disorders which are associated with an increased risk to develop BCC are: Rombo syndrome [4], Bazex-Dupre-Christol syndrome (also called Bazex syndrome or follicular atrophoderma and basal cell carcinomas) [5], Xeroderma pigmentosum [6], Muir-Torre syndrome [4], and Oculocutaneous albinism (OCA) [7].

BCC is observed in the general population typically on sun-exposed areas of the skin.

Further, an increased risk of developing BCCs exists for immunosuppression subjects or subjects that were exposed to radiation, asbestos, sun, or tanning salons. Thus, there remains a need for a non-surgical therapy for BCC that offers better treatment.

### Patidegib

Patidegib compound also known in the art as “saridegib” and “IPI-926” has the following structure:

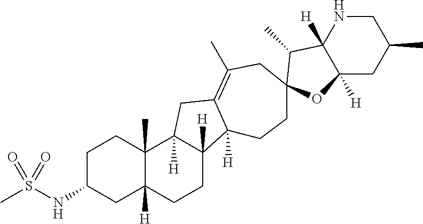

Patidegib and the process for the preparation thereof is disclosed in U.S. Pat. No. 8,785,635 (US '635) entitled “Cyclopamine analogs”. Patidegib is a member of a class of anti-cancer compounds known as hedgehog (HH) pathway inhibitors. Patidegib exhibits its pharmacological effect by inhibition of the G protein-coupled receptor smoothened, a component of the hedgehog (HH) signaling pathway. Patidegib, as prepared by the process disclosed in U.S. Pat. No. 8,785,635 (US '635) resulted with few impurities in the final product. Thus, there is a need for an improved process for the preparation of Patidegib.

### REFERENCES

[1] Yang, Xin-Hua, et al. “Inherited rare and common variants in PTCH1 and PTCH2 contributing to the predisposition to reproductive cancers.” Gene 814 (2022): 146157
[2a] Farndon P A, Del Mastro R G, Evans D G, Kilpatrick M W. Location of gene for Gorlin syndrome. Lancet 1992; 339:581.
[2b] Peris, K, et al. “Diagnosis and treatment of basal cell carcinoma: European consensus-based interdisciplinary guidelines.” European Journal of cancer 118 (2019): 10-34.[3] MacDonald DS.
[3] A systematic review of the literature of nevoid basal cell carcinoma syndrome affecting East Asians and North Europeans. Oral Surg Oral Med Oral Pathol Oral Radiol 2015; 120:396.
[4] Schierbeck J, Vestergaard T, Bygum A. Skin Cancer Associated Genodermatoses: A Literature Review. Acta Derm Venereol 2019; 99:360.
[5] Torrelo A, Sprecher E, Mediero I G, et al. What syndrome is this?Bazex-Dupre-Christol syndrome. Pediatr Dermatol 2006; 23:286.
[6] DiGiovanna J J, Kraemer K H. Shining a light on xeroderma pigmentosum. J Invest Dermatol 2012; 132:785.
[7] Kiprono S K, Chaula B M, Beltraminelli H. Histological review of skin cancers in African Albinos: a 10-year retrospective review. BMC Cancer 2014; 14:157.

### SUMMARY OF THE INVENTION

The present invention is directed to a compound of patidegib or any pharmaceutically acceptable salt thereof, wherein the compound comprises less than 0.05 wt % of impurity A

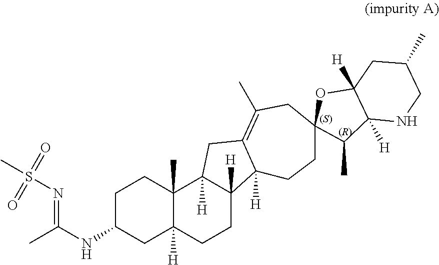

or any salt thereof, or less than 0.5 wt % of impurity B

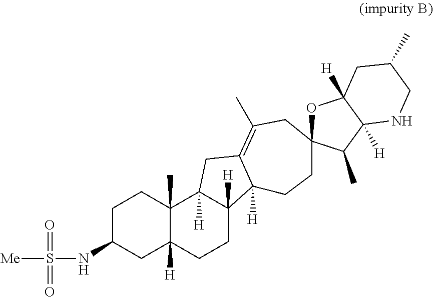

or any salt thereof, or any combination thereof. In another aspect, provided herein is a process for the preparation of patidegib compound of this invention. In another aspect, provided herein is a pharmaceutical composition comprising the patidegib compound of this invention and a pharmaceutically acceptable carrier.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 presents a synthetic scheme of a process for the preparation of a compound of patidegib of this invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In some embodiments, provided herein is a compound of patidegib or any pharmaceutically acceptable salt thereof, wherein the compound comprises less than 0.05 wt % of impurity A

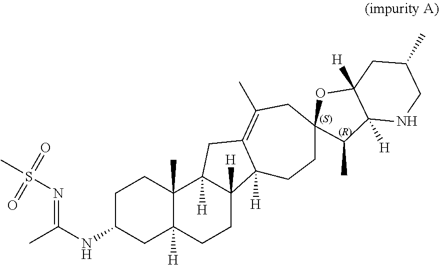

or any salt thereof, or less than 0.5 wt % of impurity B

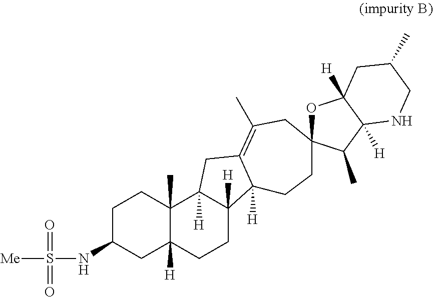

or any salt thereof, or any combination thereof. In another embodiment, the pharmaceutically acceptable salt of patidegib is HCl.

In some embodiments, provided herein is a compound of patidegib or any pharmaceutically acceptable salt thereof, wherein the compound comprises less than 0.05 wt % of impurity A

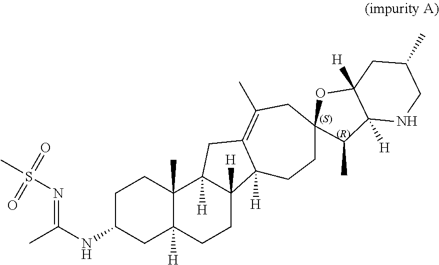

or any salt thereof. In another embodiment, the compound of patidegib comprises less than 0.2 wt %, 0.19 wt %, 0.18 wt %, 0.17 wt %, 0.16 wt %, 0.15 wt %, 0.14 wt %, 0.13 wt %, 0.12 wt %, 0.11 wt %, 0.1 wt %, 0.09 wt %, 0.08 wt %, 0.07 wt %, 0.06 wt %, 0.05 wt %, 0.04 wt %, 0.03 wt %, 0.02 wt %, 0.01 wt %, or less than the detected limit of impurity A.

In another embodiment, the compound of patidegib comprises about 0.2 wt %, 0.15 wt %, 0.1 wt %, 0.09 wt %, 0.08 wt %, 0.07 wt %, 0.06 wt %, 0.05 wt %, 0.04 wt %, 0.03 wt %, 0.02 wt %, 0.01 wt %, or less than the detected limit of impurity A. In another embodiment, the compound of patidegib does not comprise impurity A. In another embodiment, the compound of patidegib is substantially free of impurity A.

In some embodiments, provided herein is a compound of patidegib or any pharmaceutically acceptable salt thereof, wherein the compound of patidegib comprises less than 0.5 wt % of impurity B

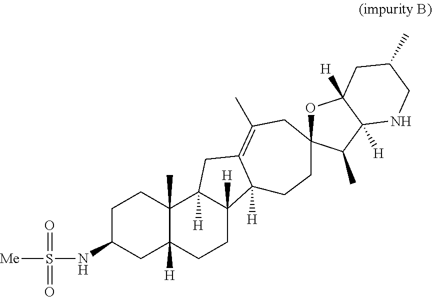

or any salt thereof. In another embodiment, the compound of patidegib comprises less than 0.8 wt %, 0.75 wt %, 0.7 wt %, 0.65 wt %, 0.6 wt %, 0.55 wt %, 0.5 wt %, 0.45 wt %, 0.4 wt %, 0.35 wt %, 0.3 wt %, 0.25 wt %, 0.2 wt %, 0.15 wt %, 0.1 wt %, 0.09 wt %, 0.08 wt %, 0.07 wt %, 0.06 wt %, 0.05 wt %, 0.04 wt %, 0.03 wt %, 0.02 wt %, 0.015 wt %, or less than the detected limit of impurity B. In another embodiment, the compound of patidegib is substantially free of impurity B.

In another embodiment, the compound of patidegib comprises less than 0.5 wt % of impurity B or any salt thereof. In another embodiment, the compound of patidegib comprises less than 0.4 wt % of impurity B or any salt thereof. In another embodiment, the compound of patidegib comprises less than 0.3 wt % of impurity B or any salt thereof. In another embodiment, the compound of patidegib comprises less than 0.2 wt % of impurity B or any salt thereof. In another embodiment, the compound of patidegib is substantially free of Impurity B.

In another embodiment, the compound of patidegib comprises about 0.8 wt %, 0.7 wt %, 0.6 wt %, 0.5 wt %, 0.45 wt %, 0.4 wt %, 0.35 wt %, 0.3 wt %, 0.25 wt %, 0.2 wt %, 0.15 wt %, 0.1 wt %, 0.09 wt %, 0.08 wt %, 0.07 wt %, 0.06 wt %, 0.05 wt %, 0.04 wt %, 0.03 wt %, 0.02 wt %, 0.015 wt %, or less than the detected limit of impurity B. In another embodiment, the compound of patidegib is substantially free of Impurity B.

In another embodiment, the compound of patidegib comprises between 0.1 wt %-0.8 wt %, 0.1 wt %-0.7 wt %, 0.1 wt %-0.6 wt %, 0.1 wt %-0.5 wt %, 0.2 wt %-0.5 wt %, 0.15 wt %-0.5 wt %, 0.05 wt %-0.5 wt %, 0.1 wt %-0.4 wt %, 0.05 wt %-0.4 wt %, 0.05 wt %-0.3 wt %, 0.05 wt %-0.2 wt %, 0.05 wt %-0.15 wt %, 0.15 wt %-0.4 wt %, 0.15 wt %-0.3 wt %, 0.15 wt %-0.25 wt %, 0.2 wt %-0.4 wt %, 0.2 wt %-0.3 wt %, 0.015-0.5 wt %, 0.015-0.4 wt %, 0.015-0.3 wt %, 0.015-0.2 wt %, 0.015-0.1 wt %, 0.015-0.05 wt % of impurity B.

In some embodiments, provided herein is a compound of patidegib or any pharmaceutically acceptable salt thereof, wherein the compound comprises less than 0.05 wt % of impurity A or any salt thereof, or less than 0.5 wt % of impurity B or any salt thereof, or any combination thereof. In another embodiment, the impurities comprise less than 0.05 wt % of impurity A or any salt thereof. In another embodiment, the compound of patidegib does not comprise impurity A. In another embodiment, the compound of patidegib is substantially free of impurity A. In another embodiment, the impurities comprise less than 0.5 wt % of impurity B or any salt thereof. In another embodiment, the impurities comprise less than 0.4 wt % of impurity B or any salt thereof. In another embodiment, the impurities comprise less than 0.3 wt % of impurity B or any salt thereof. In another embodiment, the impurities comprise less than 0.2 wt % of impurity B or any salt thereof. In another embodiment, the impurities comprise less than 0.15 wt % of impurity B or any salt thereof. In another embodiment, the impurities comprise less than 0.1 wt % of impurity B or any salt thereof. In another embodiment, the compound of patidegib is substantially free of impurity B.

In some embodiments, provided herein is a compound of patidegib or any pharmaceutically acceptable salt thereof, wherein the compound comprises less than 0.05 wt % of impurity A

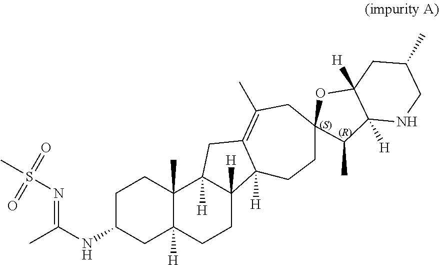

or any salt thereof, and less than 0.5 wt % of impurity B

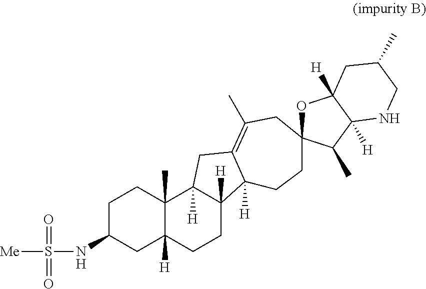

or any salt thereof.

In another embodiment, the patidegib compound provided herein does not comprise impurity A and comprises less than 0.5 wt % of impurity B.

In another embodiment, the patidegib compound provided herein is substantially free of impurity A and comprises less than 0.5 wt % of impurity B. In another embodiment, the patidegib compound provided herein comprises less than 0.05 wt % of impurity A and substantially free of impurity B.

In another embodiment, the compound of patidegib or any pharmaceutically acceptable salt thereof comprises less than 0.1 wt %, 0.09 wt %, 0.08 wt %, 0.07 wt %, 0.06 wt %, 0.05 wt %, 0.04 wt %, 0.03 wt %, 0.02 wt %, 0.01 wt %, or less than the detected limit of impurity A or any salt thereof, and less than 1 wt %, 0.9 wt %, 0.8 wt %. 0.7 wt %, 0.6 wt %, 0.5 wt %, 0.45 wt %, 0.4 wt %, 0.35 wt %, 0.3 wt %, 0.25 wt %, 0.2 wt %, 0.15 wt %, 0.1 wt %, or less than the detected limit of impurity B or any salt thereof, or any combination thereof. In another embodiment, the patidegib compound provided herein is substantially free of impurity B.

In some embodiments, provided herein is a compound of patidegib or any pharmaceutically acceptable salt thereof, wherein the compound comprises less than 0.05 wt % of impurity A or any salt thereof, and less than 0.4 wt % of impurity B or any salt thereof.

In some embodiments, provided herein is a compound of patidegib or any pharmaceutically acceptable salt thereof, wherein the compound comprises less than 0.05 wt % of impurity A or any salt thereof, and less than 0.3 wt % of impurity B or any salt thereof.

In some embodiments, provided herein is a compound of patidegib or any pharmaceutically acceptable salt thereof, wherein the compound comprises less than 0.05 wt % of impurity A or any salt thereof, and less than 0.2 wt % of impurity B or any salt thereof.

In some embodiments, provided herein is a compound of patidegib or any pharmaceutically acceptable salt thereof, wherein the compound comprises less than 0.05 wt % of impurity A or any salt thereof, or less than 0.5 wt % of impurity B or any salt thereof, or any combination thereof; and wherein the compound is at least 98% pure. In another embodiment, the compound comprises less than 0.4 wt % of impurity B or any salt thereof. In another embodiment, the compound comprises less than 0.3 wt % of impurity B or any salt thereof. In another embodiment, the compound comprises less than 0.2 wt % of impurity B or any salt thereof.

In some embodiments, provided herein is a compound of patidegib or any pharmaceutically acceptable salt thereof, wherein the compound comprises less than 0.05 wt % of impurity A or any salt thereof, and wherein the compound is at least 98% pure.

In some embodiments, provided herein is a compound of patidegib or any pharmaceutically acceptable salt thereof, wherein the compound does not comprise impurity A or any salt thereof, and wherein the compound is at least 98% pure.

In some embodiments, provided herein is a compound of patidegib or any pharmaceutically acceptable salt thereof, wherein the compound comprises less than 0.5 wt % of impurity B or any salt thereof, and wherein the compound is at least 98% pure.

In some embodiments, provided herein is a compound of patidegib or any pharmaceutically acceptable salt thereof, wherein the compound comprises less than 0.4 wt % of impurity B or any salt thereof, and wherein the compound is at least 98% pure.

In some embodiments, provided herein is a compound of patidegib or any pharmaceutically acceptable salt thereof, wherein the compound comprises less than 0.3 wt % of impurity B or any salt thereof, and wherein the compound is at least 98% pure.

In some embodiments, provided herein is a compound of patidegib or any pharmaceutically acceptable salt thereof, wherein the compound comprises less than 0.2 wt % of impurity B or any salt thereof, and wherein the compound is at least 98% pure.

In some embodiments, provided herein is a compound of patidegib or any pharmaceutically acceptable salt thereof, wherein the compound comprises less than 0.5 wt % of impurity B or any salt thereof, and does not comprise impurity A or any salt thereof, and wherein the compound is at least 98% pure.

In some embodiments, provided herein is a compound of patidegib or any pharmaceutically acceptable salt thereof, wherein the compound comprises less than 0.4 wt % of impurity B or any salt thereof, and does not comprise impurity A or any salt thereof, and wherein the compound is at least 98% pure.

In some embodiments, provided herein is a compound of patidegib or any pharmaceutically acceptable salt thereof, wherein the compound comprises less than 0.3 wt % of impurity B or any salt thereof, and does not comprise impurity A or any salt thereof, and wherein the compound is at least 98% pure.

In some embodiments, provided herein is a compound of patidegib or any pharmaceutically acceptable salt thereof, wherein the compound comprises less than 0.2 wt % of impurity B or any salt thereof, and does not comprise impurity A or any salt thereof, and wherein the compound is at least 98% pure.

In some embodiments, provided herein is a compound of patidegib or any pharmaceutically acceptable salt thereof, wherein the compound comprises less than 0.05 wt % of impurity A or any salt thereof, and less than 0.5 wt % of impurity B or any salt thereof, and wherein the compound is at least 98% pure.

In some embodiments, provided herein is a compound of patidegib or any pharmaceutically acceptable salt thereof, wherein the compound comprises less than 0.05 wt % of impurity A or any salt thereof, and less than 0.4 wt % of impurity B or any salt thereof, and wherein the compound is at least 98% pure.

In some embodiments, provided herein is a compound of patidegib or any pharmaceutically acceptable salt thereof, wherein the compound comprises less than 0.05 wt % of impurity A or any salt thereof, and less than 0.3 wt % of impurity B or any salt thereof, and wherein the compound is at least 98% pure.

In some embodiments, provided herein is a compound of patidegib or any pharmaceutically acceptable salt thereof, wherein the compound comprises less than 0.05 wt % of impurity A or any salt thereof, and less than 0.2 wt % of impurity B or any salt thereof, and wherein the compound is at least 98% pure.

In some embodiments, provided herein is a compound of patidegib or any pharmaceutically acceptable salt thereof, wherein the compound comprises less than 0.05 wt % of impurity A or any salt thereof, or less than 0.5 wt % of impurity B or any salt thereof, or any combination thereof, and wherein the compound is at least 99% pure.

In some embodiments, provided herein is a compound of patidegib or any pharmaceutically acceptable salt thereof, wherein the compound comprises less than 0.05 wt % of impurity A or any salt thereof, and wherein the compound is at least 99% pure.

In some embodiments, provided herein is a compound of patidegib or any pharmaceutically acceptable salt thereof, wherein the compound does not comprise impurity A or any salt thereof, and wherein the compound is at least 99% pure.

In some embodiments, provided herein is a compound of patidegib or any pharmaceutically acceptable salt thereof, wherein the compound comprises less than 0.5 wt % of impurity B or any salt thereof, and wherein the compound is at least 99% pure.

In some embodiments, provided herein is a compound of patidegib or any pharmaceutically acceptable salt thereof, wherein the compound comprises less than 0.4 wt % of impurity B or any salt thereof, and wherein the compound is at least 99% pure.

In some embodiments, provided herein is a compound of patidegib or any pharmaceutically acceptable salt thereof, wherein the compound comprises less than 0.3 wt % of impurity B or any salt thereof, and wherein the compound is at least 99% pure.

In some embodiments, provided herein is a compound of patidegib or any pharmaceutically acceptable salt thereof, wherein the compound comprises less than 0.2 wt % of impurity B or any salt thereof, and wherein the compound is at least 99% pure.

In some embodiments, provided herein is a compound of patidegib or any pharmaceutically acceptable salt thereof, wherein the compound comprises less than 0.05 wt % of impurity A or any salt thereof, and less than 0.5 wt % of impurity B or any salt thereof, or any combination thereof; and wherein the compound is at least 99% pure.

In some embodiments, provided herein is a compound of patidegib or any pharmaceutically acceptable salt thereof, wherein the compound comprises less than 0.05 wt % of impurity A or any salt thereof, and less than 0.4 wt % of impurity B or any salt thereof, or any combination thereof; and wherein the compound is at least 99% pure.

In some embodiments, provided herein is a compound of patidegib or any pharmaceutically acceptable salt thereof, wherein the compound comprises less than 0.05 wt % of impurity A or any salt thereof, and less than 0.3 wt % of impurity B or any salt thereof, or any combination thereof; and wherein the compound is at least 99% pure.

In some embodiments, provided herein is a compound of patidegib or any pharmaceutically acceptable salt thereof, wherein the compound comprises less than 0.05 wt % of impurity A or any salt thereof, and less than 0.2 wt % of impurity B or any salt thereof, or any combination thereof, and wherein the compound is at least 99% pure.

In some embodiments, provided herein is a compound of patidegib or any pharmaceutically acceptable salt thereof, wherein the compound does not comprise impurity A or any salt thereof, and less than 0.5 wt % of impurity B or any salt thereof, or any combination thereof, and wherein the compound is at least 99% pure.

In some embodiments, provided herein is a compound of patidegib or any pharmaceutically acceptable salt thereof, wherein the compound does not comprise impurity A or any salt thereof, and less than 0.4 wt % of impurity B or any salt thereof, or any combination thereof, and wherein the compound is at least 99% pure.

In some embodiments, provided herein is a compound of patidegib or any pharmaceutically acceptable salt thereof, wherein the compound does not comprise impurity A or any salt thereof, and less than 0.3 wt % of impurity B or any salt thereof, or any combination thereof; and wherein the compound is at least 99% pure.

In some embodiments, provided herein is a compound of patidegib or any pharmaceutically acceptable salt thereof, wherein the compound does not comprise impurity A or any salt thereof, and less than 0.2 wt % of impurity B or any salt thereof, or any combination thereof; and wherein the compound is at least 99% pure.

In some embodiment, the present invention provides a compound of patidegib or any pharmaceutically acceptable salt thereof, wherein the compound is at least 98% pure.

In some embodiment, the present invention provides a compound of patidegib or any pharmaceutically acceptable salt thereof, wherein the compound is at least 99% pure.

In some embodiment, the present invention provides a compound of patidegib or any pharmaceutically acceptable salt thereof, wherein the compound is substantially pure.

### Process

In some embodiments, provided herein is an improved process for the preparation of patidegib. In the process for the preparation of patidegib as disclosed in U.S. Pat. No. 8,785,635 (US '635) the final product (patidegib) comprised at least 0.05 wt % of impurity A and at least 0.5 wt % of impurity B as shown in Example 2. The process described in US '635 cannot be scaled up above 1 kg, because the CBz intermediates are not crystalline and cannot be isolated. Therefore, the process of US '635 utilizes distillations to dryness. Further, the process of US '635 employs hazardous and cryogenic conditions (K-selectride mediated selective reduction) and purification by column chromatography.

In contrast, the process provided herein comprises Bn-protected intermediates which are surprisingly crystalline solids which allow better reproducibility and provides a scaled-up process. In some embodiments, the process provided herein is scaled up beyond 1 kg batch size.

In some embodiments, provided herein is a process for the preparation of a compound of patidegib or any pharmaceutically acceptable salt thereof of this invention, wherein the process comprises the following steps:
a′) preparing Compound 2

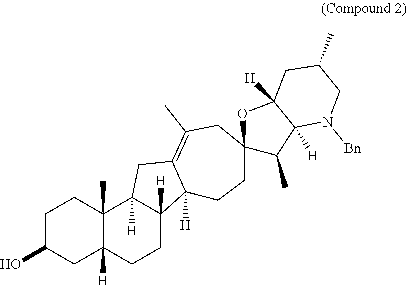

from Compound 1

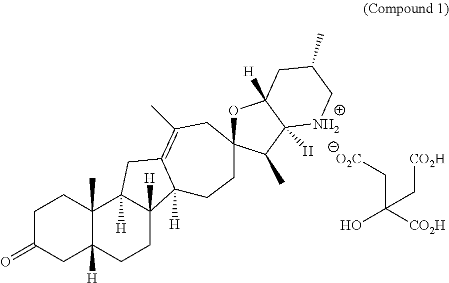

by the process provided in U.S. Pat. No. 9,879,025 incorporated herein by reference, comprising benzylation and reduction with Ru catalyst;
a) preparing Compound 3

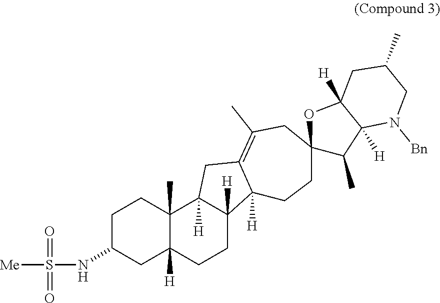

from Compound 2

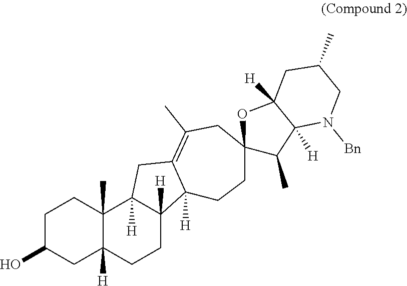

wherein the alcohol group of Compound 2 undergoes mesylation of the alcohol group, followed by substitution of the mesylate group to an azide group, followed by reduction of the azide group into an amine group, and followed by mesylation of the amine group to obtain Compound 3;
b) deprotecting of the benzyl group of Compound 3; and
c) adding acid to obtain a salt of the compound of patidegib of this invention.

In some embodiments, provided herein is a process for the preparation of a compound of patidegib or any pharmaceutically acceptable salt thereof, wherein the compound comprises less than 0.05 wt % of impurity A

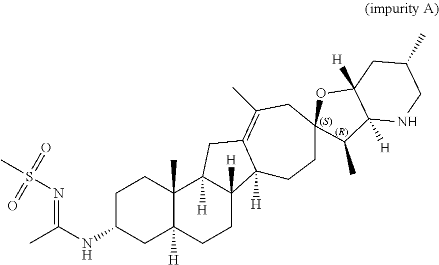

or any salt thereof, or less than 0.5 wt % of impurity B

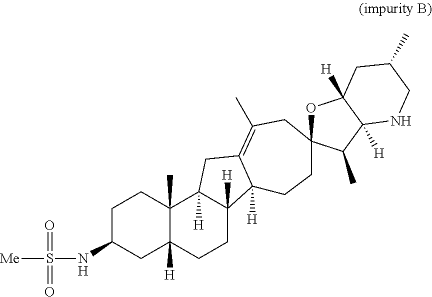

or any salt thereof, or any combination thereof, wherein the process comprises the following steps:
a′) preparing Compound 2

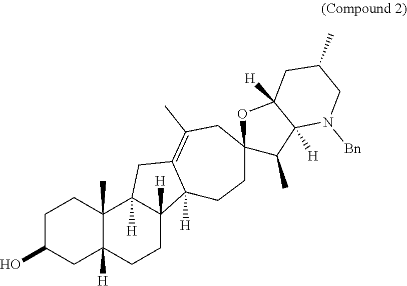

from Compound 1

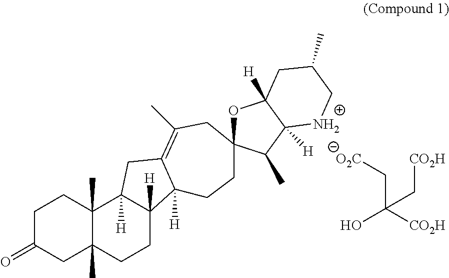

by the process provided in U.S. Pat. No. 9,879,025 incorporated herein by reference, comprising benzylation and reduction with Ru catalyst;
a) preparing Compound 3

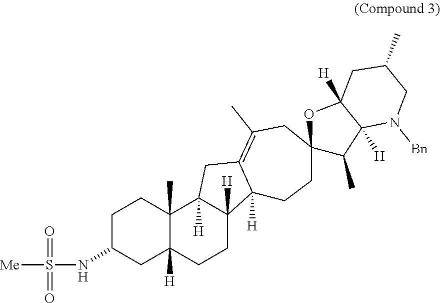

from Compound 2

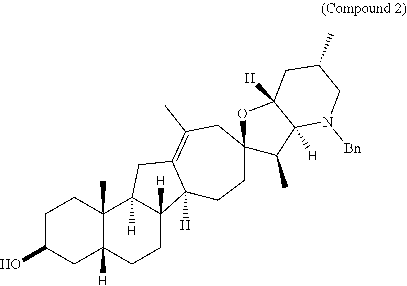

wherein the alcohol group of Compound 2 undergoes mesylation of the alcohol group, followed by substitution of the mesylate group to an azide group, followed by reduction of the azide group into an amine group, and followed by mesylation of the amine group to obtain Compound 3;
b) deprotecting of the benzyl group of Compound 3; and
c) adding acid to obtain a salt of the compound of patidegib of this invention.

In another embodiment, the benzylation of step (a′), comprises the use of Bn-halide and a base. In another embodiment, the benzylation comprises the use of BnBr, NaOH and 2-Me-THF.

In another embodiment, the reduction of the ketone of Compound 1 to the alcohol of Compound 2 comprises the use of Ru catalyst/2-(N-ethylamino)-2 methyl-propan-1-ol and a base. In another embodiment, the reduction of the ketone of Compound 1 to the alcohol of Compound 2 comprises the use of Ru catalyst/2-(N-ethylamino)-2 methyl-propan-1-ol, KOiPr and 2-Me-THF and isopropyl alcohol (IPA). In another embodiment, the reduction of the ketone of Compound 1 to the alcohol of Compound 2 comprises the use of Ru catalyst/ephedrine and a base. In another embodiment, the reduction of the ketone of Compound 1 to the alcohol of Compound 2 comprises the use of Ru catalyst/ephedrine, KOiPr and 2-Me-THF and IPA.

In another embodiment, Compound 2 obtained in step (a′) is further crystallized.

In some embodiments, provided herein is a process for the preparation of a compound of patidegib or any pharmaceutically acceptable salt thereof, wherein the compound comprises less than 0.05 wt % of impurity A

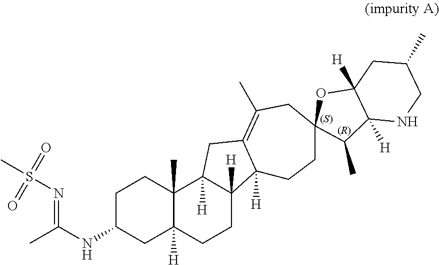

or any salt thereof, or less than 0.5 wt % of impurity B

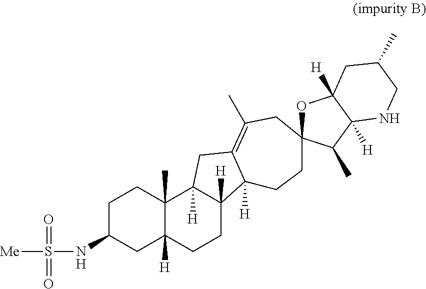

or any salt thereof, or any combination thereof, wherein the process comprises the following steps:
a) preparing Compound 3

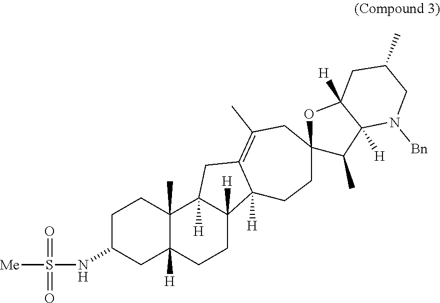

from Compound 2

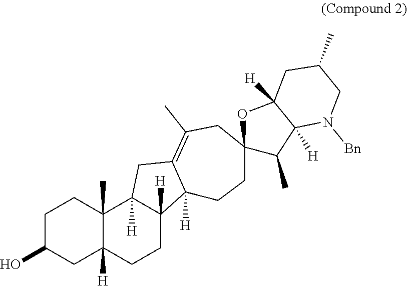

wherein the alcohol group of Compound 2 undergoes mesylation of the alcohol group, followed by substitution of the mesylate group to an azide group, followed by reduction of the azide group into an amine group, and followed by mesylation of the amine group to obtain Compound 3;
b) deprotecting of the benzyl group of Compound 3; and
c) adding acid to obtain a salt of the compound of patidegib of this invention.

In another embodiment, the mesylation of the alcohol group of Compound 2 in step (a) comprises the use of methanesulfonyl chloride (MsCl) and a base. In another embodiment, the base in the mesylation step of the alcohol group is a tertiary amine. In another embodiment, the tertiary amine comprises triethyl amine, diisopropylethyl amine, N-methyl morpholine, or pyridine. In another embodiment, the base in the mesylation step of the alcohol group is triethyl amine. In another embodiment, the mesylation of the alcohol group of Compound 2 in step (a) comprises the use of a solvent, MsCl and a base. In another embodiment, the solvent is 2-Me-THF.

In another embodiment, the substitution of the mesylate group into an azide group in step (a) refers to a displacement of the mesylate into an azide group. In another embodiment, the substitution of the mesylate group into an azide group in step (a) comprises the use of quaternary phosphonium azide, quaternary ammonium azide, or sodium azide. In another embodiment, the quaternary phosphonium azide comprises tetra-n-butylphosphonium azide (TBPA), or hexadecyltributylphosphonium azide. In another embodiment, the quaternary ammonium azide is tetrabutylammonium azide (TBAA).

In another embodiment, the substitution of the mesylate group into an azide group in step (a) comprises the use of tetra-n-butylphosphonium azide (TBPA) or tetrabutylammonium azide (TBAA). In another embodiment, the substitution of the mesylate group into an azide group in step (a) comprises the use of tetra-n-butylphosphonium azide (TBPA). In another embodiment, the substitution of the mesylate group into an azide group in step (a) comprises the use of tetrabutylammonium azide (TBAA). In another embodiment, the substitution of the mesylate group into an azide group in step (a) comprises the use of sodium azide.

In another embodiment, the reduction of the azide group into an amine group in step (a) comprises catalytic hydrogenation followed by acid/base extractions. In another embodiment, the catalytic hydrogenation of the azide into an amine comprises the use of platinum hydroxide, Pd/C, Pt/C or Ni/C catalyst with H₂, or lithium aluminum hydride (LiAlH₄). Each represents a separate embodiment of this invention. In another embodiment, the catalytic hydrogenation of the azide into an amine comprises the use of Pt/C or Ni/C catalyst with H₂. In another embodiment, the catalytic hydrogenation is performed in 1-5 atm of hydrogen. In another embodiment, the catalytic hydrogenation is performed in 2-4 atm of hydrogen. In another embodiment, the catalytic hydrogenation is performed in 2.5±0.5 atm of hydrogen. In another embodiment, the catalytic hydrogenation is performed in 3 atm of hydrogen.

In another embodiment, the catalytic hydrogenation comprises the use of Pt/C with H₂. In another embodiment, the catalytic hydrogenation comprises the use of Ni/C catalyst with H₂. In another embodiment, the acid/base extractions to remove the process impurities following the azide hydrogenation comprise a strong acid and an inorganic base. In another embodiment, the strong acid comprises HCl, H₂SO₄, or H₃PO₄. Each represents a separate embodiment of this invention. In another embodiment, the inorganic base comprises NaOH, KOH, LiOH, CsOH, or K₃PO₄. Each represents a separate embodiment of this invention. In another embodiment, the acid/base extractions to remove the process impurities following the azide hydrogenation in step (a) comprise HCl, 2-MeTHF and NaOH.

In another embodiment, the mesylation of the amine group in step (a) comprises the use of methanesulfonyl chloride (MsCl) and a base. In another embodiment, the base of the mesylation of the amine group in step (a) comprises tertiary amine, sodium carbonate, or trisodium phosphate. In another embodiment, the mesylation of the amine group in step (a) comprises the use of a solvent, MsCl and a base.

In another embodiment, the solvent of the mesylation of the amine group comprises 2-Me-THF, THF, dichloromethane (DCM), toluene, or any combination thereof. Each represents a separate embodiment of this invention. In another embodiment, the solvent is 2-Me-THF.

In another embodiment, Compound 3 obtained in step (a) is further crystallized. In another embodiment, Compound 3 is crystallized with IPA.

In another embodiment, Compound 3 is isolated by crystallization. In another embodiment, the crystallization of Compound 3, is done with IPA.

In another embodiment, the deprotecting of the benzyl group in step (b) comprises catalytic hydrogenation. In another embodiment, the catalytic hydrogenation of the benzyl group in step (b) comprises the use of Pd/C H₂. In another embodiment, the catalytic hydrogenation further comprises crystallization with isopropyl alcohol (IPA). In another embodiment, the catalytic hydrogenation comprises the use of Pd/C H₂ in 2-Me-THF and isopropyl alcohol (IPA).

In another embodiment, the acid used in step (c) to obtain a salt of the compound of patidegib is HCl. In another embodiment, the acid used in step (c) comprises HCl and isopropyl alcohol (IPA).

In some embodiment, the process provided herein comprises the use of 2-Me-THF as a sole solvent or a mixture of 2-Me-THF with additional solvent in all steps.

In some embodiments, impurity B is formed from Compound (1), as presented in the following scheme 1:

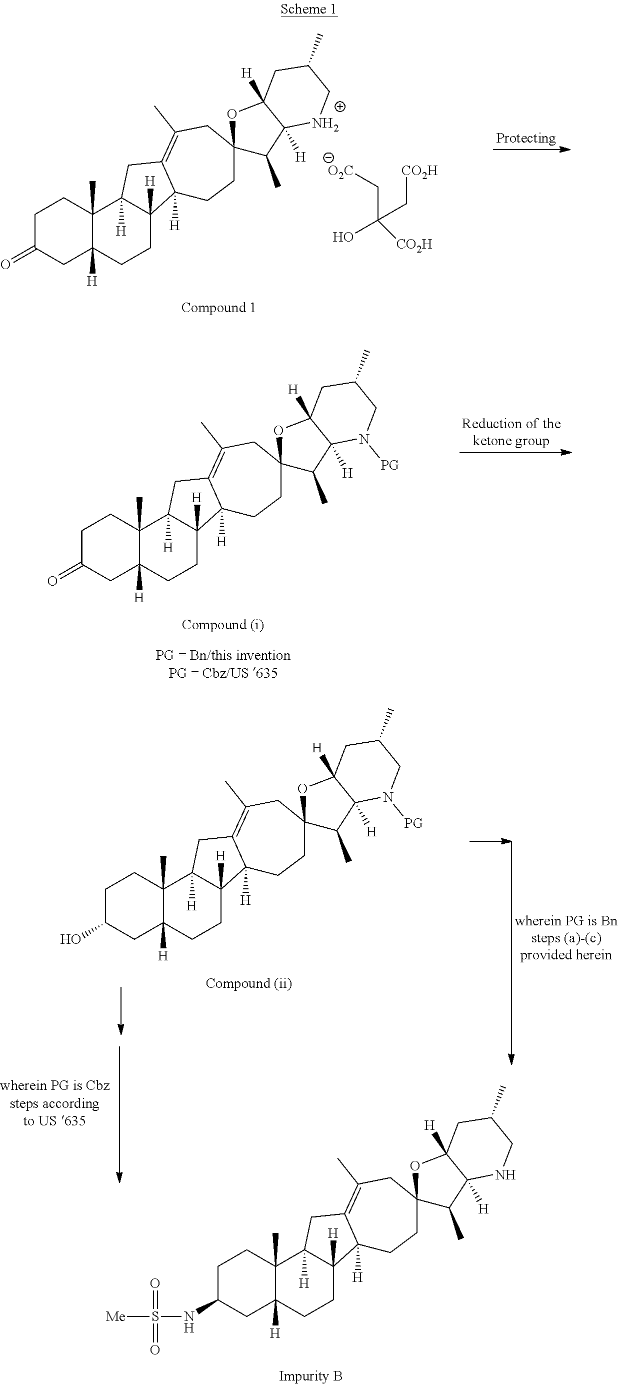

In some embodiments, impurity A originates in the process for the preparation of patidegib covered by U.S. Pat. No. 8,785,635 (US '635).

In some embodiments, provided herein is a compound of patidegib or any acceptable salt thereof, comprising less than 0.05 wt % of impurity A

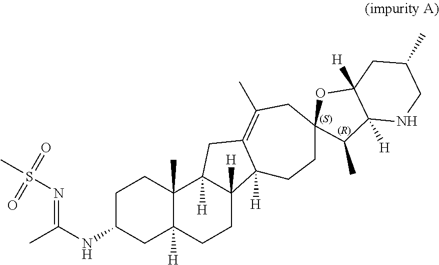

or any salt thereof, less than 0.5 wt % of impurity B

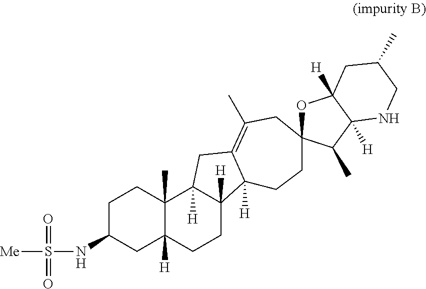

or any salt thereof, or any combination thereof, prepared according to the process provided herein. In some embodiments, provided herein is a compound of patidegib or any acceptable salt thereof, comprises less than 0.5 wt % of impurity B

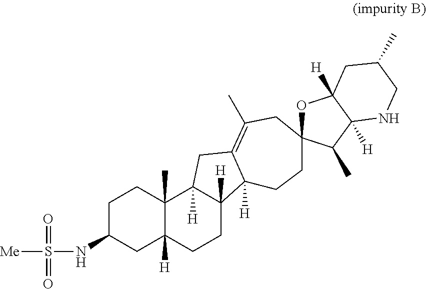

or any salt thereof, and the patidegib does not comprise impurity A

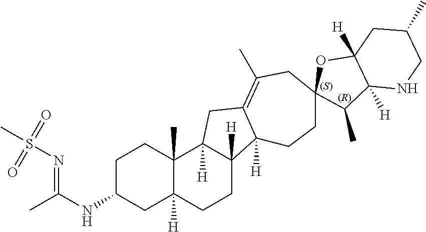

or any salt thereof, wherein the compound of patidegib is prepared according to the process provided herein.

### Pharmaceutical Composition

In some embodiment, provided herein is a pharmaceutical composition comprising the compound of patidegib of this invention, and a pharmaceutically acceptable carrier.

In some embodiment, provided herein is a pharmaceutical composition comprising the compound of patidegib of this invention prepared according to the process of this invention, and a pharmaceutically acceptable carrier.

In some embodiments, provided herein is a pharmaceutical composition comprising a compound of patidegib or any pharmaceutically acceptable salt thereof, wherein the compound comprises less than 0.05 wt % of impurity A or any salt thereof, or less than 0.5 wt % of impurity B or any salt thereof, or any combination thereof, and a pharmaceutically acceptable carrier.

In some embodiments, provided herein is a pharmaceutical composition comprising a compound of patidegib or any pharmaceutically acceptable salt thereof, wherein the compound comprises less than 0.05 wt % of impurity A or any salt thereof, or less than 0.5 wt % of impurity B or any salt thereof, or any combination thereof, prepared according to the process provided herein, and a pharmaceutically acceptable carrier.

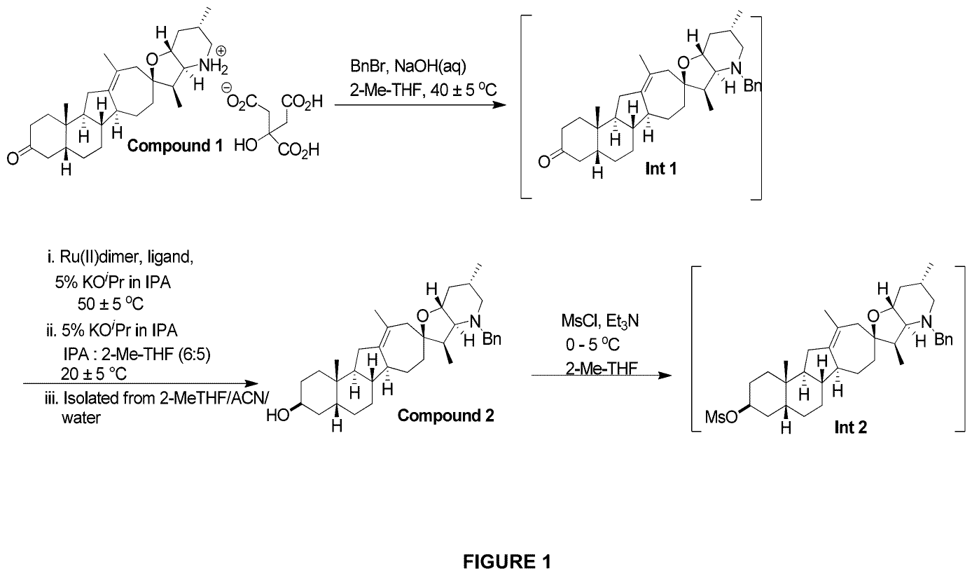

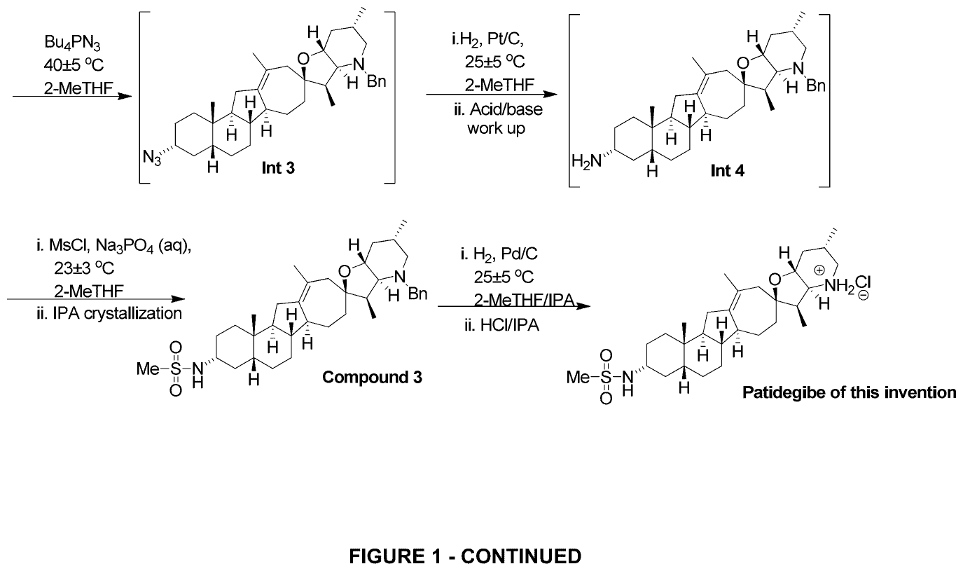

In some embodiments, provided herein is a pharmaceutical composition comprising a compound of patidegib or any pharmaceutically acceptable salt thereof, wherein the compound comprises less than 0.05 wt % of impurity A or any salt thereof, or less than 0.5 wt % of impurity B or any salt thereof, or any combination thereof, prepared according to the process provided in FIG. 1 and/or Example 1, and a pharmaceutically acceptable carrier.

In some embodiments, provided herein is a pharmaceutical composition comprising a compound of patidegib or any pharmaceutically acceptable salt thereof, wherein the compound comprises does not comprise impurity A or any salt thereof, and less than 0.5 wt % of impurity B or any salt thereof, prepared according to the process provided herein, and a pharmaceutically acceptable carrier.

In some embodiments, provided herein is a pharmaceutical composition comprising a compound of patidegib or any pharmaceutically acceptable salt thereof, wherein the compound comprises does not comprise impurity A or any salt thereof, and less than 0.5 wt %, 0.4 wt %, 0.3 wt % or less than 0.2 wt % of impurity B or any salt thereof, prepared according to the process provided herein, and a pharmaceutically acceptable carrier. In another embodiment, the pharmaceutical composition provided herein comprises less than 0.05 wt % of impurity A and substantially free of impurity B.

In some embodiments, the pharmaceutical composition of this invention comprises patidegib in an amount of about 0.1 wt % to about 6 wt %. In another embodiment, the composition comprises patidegib in an amount of about 0.1-2 wt %, 0.1-3 wt %, 0.1-4%, 0.1-5 wt %, 1-2 wt %, 1.5-3 wt %, 1-3 wt %, 1-4 wt % w/w, 1-5 wt %, 2-4 wt %, 2-5 wt %, 2-6 wt %, or 0.1-6 wt %. In another embodiment, the composition comprises patidegib in an amount of about 0.1 wt %, 0.5 wt %, 1 wt %, 1.5 wt %, 2 wt %, 2.5 wt %, 3 wt %, 3.5 wt %, 4 wt %, 4.5 wt %, 5 wt %, 5.5 wt %, or 6 wt %. In another embodiment, the composition comprises patidegib in an amount of about 2 wt %, 3 wt %, or 4 wt %. In another embodiment, the composition comprises patidegib in an amount of about 2 wt %.

In some embodiments, the pharmaceutical composition of this invention is selected from a cream, an ointment, a gel, a lotion, a spray, a patch, or a foam. Each represents a separate embodiment of this invention. In some embodiments, the pharmaceutical composition of this invention is formulated as a gel.

In another embodiment, the compound of patidegib of this invention, or a pharmaceutically acceptable salt thereof is formulated with any pharmaceutically acceptable carrier, and may be a liquid, semi-solid or a solid composition. Pharmaceutical carriers or vehicles suitable for topical administration of the compositions to the skin or mucosa are known to those skilled in the art and the patidegib compound can be included in the carrier in an amount sufficient to provide a therapeutically useful effect in the treatment of and/or prevention of BCC. In one embodiment, the composition comprising a compound of patidegib of this invention, or a pharmaceutically acceptable salt thereof, is a liquid. Liquid dosage forms for topical administration include emulsions, solutions and suspensions containing diluents commonly used in the art, such as alcohols, glycols, oils, water and the like. The compositions may also include wetting agents, emulsifying and suspending agents.

The composition may be in the form of solutions, suspensions, emulsions, ointments, lotions, gels, and the like. Emulsions of the form oil-in-water or water-in-oil are contemplated. Gels are formed by the entrapment of large amounts of aqueous or aqueous-alcoholic liquids in a network of polymers or of colloidal solid particles. Such polymers or colloids are typically present at concentrations of less than 10 wt % and are also referred to as gelling agents or thickening agents. Examples of suitable gelling agents include carboxymethyl cellulose, hydroxypropylmethyl cellulose, hydroxyethyl cellulose, hydroxypropyl cellulose, methyl cellulose, sodium alginate, alginic acid, pectin, tragacanth, carrageen, agar, clays, aluminum silicate, carbomers, etc.

Creams and ointments may also be utilized. They are emulsions of oleaginous substances and water (i.e. the carrier). The cream may be a water-in-oil (w/o) in which an aqueous phase is dispersed in an oil phase, or an oil-in-water (o/w) which has an oil dispersed within an aqueous base. An ointment is also contemplated and is typically more viscous than an oil-in-water cream. Traditional ointment bases (i.e. the carrier) include hydrocarbons (petrolatum, beeswax, etc.) vegetable oils, fatty alcohols (cholesterol, lanolin, wool alcohol, stearyl alcohol, etc.) or silicones. Pastes are a type of ointment into which a high percentage of insoluble particulate solids have been added, up to 50% by weight. Insoluble solids such as starch, zinc oxide, calcium carbonate, or talc may be used.

Aerosols may also be utilized. The compound may be dissolved in a propellant and a co-solvent such ethanol, acetone, hexadecyl alcohol, etc. Foaming agents may be incorporated to produce a mousse.

Emollient or lubricating vehicles that help hydrate the skin can also be used. Examples of suitable bases or vehicles for preparing hydrating compositions for use with human skin are petrolatum, petrolatum plus volatile silicones, lanolin, cold cream (USP), and hydrophilic ointment (USP).

A wide variety of methods may be used for preparing the formulations described above. The formulations may be prepared by combining together the components of the formulation, as described herein, at a temperature and for a time sufficient to provide a pharmaceutically acceptable composition. The term “combining together”, as used herein, means that all of the components of the compositions may be combined and mixed together at about the same time. The term “combining together” also means that the various components may be combined in one or more sequences to provide the desired product. The formulation can be prepared on a weight/weight (w/w) or a weight/volume (w/v) basis depending upon the form of the final dosage form.

The composition comprises a weight fraction of patidegib or a pharmaceutically acceptable salt thereof, that may be dissolved, suspended, dispersed or otherwise mixed in a selected carrier or vehicle, at an effective concentration such that the pruritic condition is relieved or ameliorated. Compositions that are in solution form and intended for topical administration may contain an amount of patidegib or a pharmaceutically acceptable salt thereof, between about 0.1 wt % to about 6 wt %, with the balance of the solution being water, a suitable organic solvent or other suitable solvent or buffer. Compositions that are formulated as solutions, emulsions, or suspensions can be applied to the skin, or can be formulated as an aerosol or foam and applied to the skin as a spray-on. The aerosol compositions typically contain from 25 wt % to 80 wt %, preferably from 30 wt % to 50 wt %, of a suitable propellant.

Compositions of solid forms intended for topical application can be formulated as stick-type compositions intended for application to the lips or other parts of the body. Such compositions contain an effective amount of patidegib or a pharmaceutically acceptable salt thereof. The amount of the patidegib compound is typically from about 0.1 wt % to about 6 wt %. The solid form of the composition may also contain from about 40 wt % to 98 wt %, preferably from about 50 wt % to 90 wt %, of carrier(s).

In some embodiments, the pharmaceutically acceptable carrier of the composition of this invention comprise DGME (diethylene glycol monoethyl ether), HPC (hydroxypropyl cellulose), borate buffer, dehydrated alcohol, propylene glycol, phenoxyethanol, or any combination thereof. In another embodiment, the pharmaceutically acceptable carrier of the composition of this invention encompasses carriers, excipients, and diluents, meaning a material, composition or vehicle, such as a liquid or solid filler, diluent, excipient, solvent or encapsulating material involved in carrying or transporting a pharmaceutical agent across the stratum corneum.

### Method of Use

In some embodiments, provided herein is a method of treatment of basal cell carcinoma (BCC) lesions, comprising topically administering, to a subject in need, the pharmaceutical composition provided herein.

In some embodiments, provided herein is a method of preventing, delaying and/or inhibiting the formation of new basal cell carcinoma in a subject, comprising topically administering to a subject the pharmaceutical composition provided herein.

In some embodiments, provided herein is a method of treatment of basal cell carcinoma (BCC) lesions and/or preventing, delaying and/or inhibiting the formation of new BCC lesions, comprising topically administering a pharmaceutical composition provided herein, to a subject in need thereof, wherein the subject has at least one of (i) at least 1 BCC lesion; (ii) a genetic mutation PTCH; or (iii) at least one disorder comprising non-melanoma skin cancer, Rombo syndrome, Bazex-Dupre-Christol syndrome, Xeroderma pigmentosum, Muir-Torre syndrome, Oculocutaneous albinism, Gorlin syndrome, or any combination thereof. In another embodiment, said subject has at least 1 BCC lesion. In another embodiment, said subject has a genetic mutation PTCH. In another embodiment, said subject has at least one disorder comprising non-melanoma skin cancer, Rombo syndrome, Bazex-Dupre-Christol syndrome, Xeroderma pigmentosum, Muir-Torre syndrome, Oculocutaneous albinism, Gorlin syndrome, or any combination thereof. In another embodiment, said subject has non-melanoma skin cancer. In another embodiment, said subject has Rombo syndrome. In another embodiment, said subject has Bazex-Dupre-Christol syndrome. In another embodiment, said subject has Xeroderma pigmentosum. In another embodiment, said subject has Muir-Torre syndrome. In another embodiment, said subject has Oculocutaneous albinism. In another embodiment, said subject has Gorlin syndrome.

In some embodiments, provided herein is a method of treatment of basal cell carcinoma (BCC) lesions and/or preventing, delaying and/or inhibiting the formation of new BCC lesions, comprising topically administering a pharmaceutical composition provided herein, to a subject in need thereof, wherein the subject has a genetic mutation PTCH1. In another embodiment, the subject suffers from Gorlin Syndrome.

In some embodiments, provided herein is a method of treatment of basal cell carcinoma (BCC) lesions and/or preventing, delaying and/or inhibiting the formation of new BCC lesions, in a subject with Gorlin syndrome comprising topically administering a pharmaceutical composition provided herein, to a subject in need thereof, wherein the subject has a genetic mutation PTCH1.

In some embodiments, provided herein is a method of treatment of basal cell carcinoma (BCC) lesions and/or preventing, delaying and/or inhibiting the formation of new BCC lesions, comprising topically administering a pharmaceutical composition of this invention, to a subject in need thereof, wherein the subject has at least one of (i) at least 1 BCC lesions; or (ii) a genetic mutation PTCH; or (iii) at least 1 BCC lesions and a genetic mutation PTCH; and wherein said subject suffers from Gorlin syndrome. In another embodiment, the BCC is facial BCC.

In some embodiments, provided herein is a method of treatment of basal cell carcinoma (BCC) lesions and/or preventing, delaying and/or inhibiting the formation of new BCC lesions, comprising topically administering a pharmaceutical composition of this invention, to a subject in need thereof, wherein the subject has at least one of (i) at least 6 facial BCC lesions; or (ii) a genetic mutation PTCH; or (iii) at least 6 BCC lesions and a genetic mutation PTCH; and wherein said subject suffers from Gorlin syndrome.

In some embodiments, provided herein is a method of treatment of basal cell carcinoma (BCC) lesions and/or preventing, delaying and/or inhibiting the formation of new basal cell carcinoma lesions, comprising topically administering a pharmaceutical composition provided herein, to a subject in need thereof, wherein the subject has at least one of (i) at least 6 BCC lesions; or (ii) a genetic mutation; or (iii) at least 6 BCC lesions and a genetic mutation. In another embodiment, the genetic mutation comprises PTCH, PTCH1, PTCH2, SMO, SUFU or any combination thereof.

In some embodiments, provided herein is a method of treatment of basal cell carcinoma (BCC) lesions and/or preventing, delaying and/or inhibiting the formation of new basal cell carcinoma lesions, comprising topically administering a pharmaceutical composition provided herein, to a subject in need thereof, wherein the subject has at least one of (i) at least 6 BCC lesions; or (ii) a genetic mutation PTCH; or (iii) at least 6 BCC lesions and a genetic mutation PTCH. In another embodiment, said PTCH is PTCH1. In another embodiment, said PTCH is PTCH2. In another embodiment, said PTCH is PTCH1 and/or PTCH2. In another embodiment, said BCC lesions are facial BCC lesions. In another embodiment, said subject must have at least 8 BCC lesions. In another embodiment, said subject must have at least 10 BCC lesions. In another embodiment, said subject must have at least 12 BCC lesions. In another embodiment, said subject must have at least 4 facial BCC lesions. In another embodiment, said subject must have at least 6 facial BCC lesions. In another embodiment, said subject must have at least 8 facial BCC lesions. In another embodiment, said subject must have at least 10 facial BCC lesions. In another embodiment, said subject must have at least 12 facial BCC lesions.

In some embodiments, provided herein is a method of treatment of basal cell carcinoma (BCC) lesions and/or preventing, delaying and/or inhibiting the formation of new basal cell carcinoma lesions, comprising topically administering a pharmaceutical composition provided herein, to a subject in need thereof, wherein the subject has at least one of (i) least 6 BCC lesions; or (ii) a genetic mutation PTCH; or (iii) the subject must have at least 6 BCC lesions and a genetic mutation PTCH, and wherein the composition of this invention comprises patidegib in an amount of about 0.1 wt % to about 6 wt %. In another embodiment, the composition comprises patidegib in an amount of about 0.1-2 wt %, 0.1-3 wt %, 0.1-4 wt %, 0.1-5 wt %, 1-2 wt %, 1.5-3 wt %, 1-3 wt %, 1-4 wt %, 1-5 wt %, 2-4 wt %, 2-5 wt %, 2-6 wt %, or 0.1-6 wt %. In another embodiment, the composition comprises patidegib in an amount of about 1 wt %, 2 wt %, 3 wt %, 4 wt %, 5 wt %, or 6 wt %. In another embodiment, the composition comprises patidegib in an amount of about 2 wt %, 3 wt %, or 4 wt %. In another embodiment, the composition comprises patidegib in an amount of about 2 wt %.

In some embodiments, provided herein is a method of treatment of basal cell carcinoma (BCC) lesions and/or preventing, delaying and/or inhibiting the formation of new basal cell carcinoma lesions, comprising topically administering a pharmaceutical composition provided herein, to a subject in need thereof, wherein the subject has at least one of (i) at least 6 facial BCC lesions; or (ii) a genetic mutation PTCH; or (iii) at least 6 BCC lesions and a genetic mutation PTCH. In another embodiment, said PTCH is PTCH1. In another embodiment, said PTCH is PTCH2. In another embodiment, said PTCH is PTCH1 and/or PTCH2. In another embodiment, said BCC lesions are facial BCC lesions.

In another embodiment, said subject must have at least 8 facial BCC lesions. In another embodiment, said subject must have at least 10 facial BCC lesions. In another embodiment, said subject must have at least 12 facial BCC lesions. In another embodiment, the subject must have at least 8 BCC lesions and a genetic mutation PTCH. In another embodiment, the subject must have at least 10 BCC lesions and a genetic mutation PTCH. In another embodiment, the subject must have at least 12 BCC lesions and a genetic mutation PTCH.

In another embodiment, the subject must have at least 6 facial BCC lesions and a genetic mutation PTCH. In another embodiment, the subject must have at least 8 facial BCC lesions and a genetic mutation PTCH. In another embodiment, the subject must have at least 10 facial BCC lesions and a genetic mutation PTCH. In another embodiment, the subject must have at least 12 facial BCC lesions and a genetic mutation PTCH.

In some embodiments, provided herein is a method of treatment of basal cell carcinoma (BCC) lesions and/or preventing, delaying and/or inhibiting the formation of new basal cell carcinoma lesions, comprising topically administering a pharmaceutical composition provided herein, to a subject in need thereof, wherein the subject has at least one of (i) at least 6 facial BCC lesions; or (ii) a genetic mutation PTCH; or (iii) at least 6 facial BCC lesions and a genetic mutation PTCH. In some embodiments, provided herein is a method of treatment of basal cell carcinoma (BCC) lesions and/or preventing, delaying and/or inhibiting the formation of new basal cell carcinoma lesions, comprising topically administering a pharmaceutical composition provided herein, to a subject in need thereof, wherein the subject must have at least 6 BCC lesions.

In some embodiments, provided herein is a method of treatment of basal cell carcinoma (BCC) lesions and/or preventing, delaying and/or inhibiting the formation of new basal cell carcinoma lesions, comprising topically administering a pharmaceutical composition provided herein, to a subject in need thereof, wherein the subject must have at least 6 facial BCC lesions.

In some embodiments, provided herein is a method of treatment of basal cell carcinoma (BCC) lesions and/or preventing, delaying and/or inhibiting the formation of new basal cell carcinoma lesions, comprising topically administering a pharmaceutical composition provided herein, to a subject in need thereof, wherein the subject must have a genetic mutation PTCH.

In some embodiments, provided herein is a method of treatment of basal cell carcinoma (BCC) lesions and/or preventing, delaying and/or inhibiting the formation of new basal cell carcinoma lesions, comprising topically administering a pharmaceutical composition provided herein, to a subject in need thereof, wherein the subject must have at least 6 BCC lesions and a genetic mutation PTCH.

In some embodiments, provided herein is a method of treatment of basal cell carcinoma (BCC) lesions and/or preventing, delaying and/or inhibiting the formation of new basal cell carcinoma lesions, comprising topically administering a pharmaceutical composition provided herein, to a subject in need thereof, wherein the subject must have at least 6 facial BCC lesions and a genetic mutation PTCH.

In some embodiments, provided herein is a method of treatment of basal cell carcinoma (BCC), comprising topically administering a pharmaceutical composition of this invention, to a subject in need thereof, wherein the subject eligible for the treatment is selected according to any one of the following criteria (i) the subject must have at least 6 BCC lesions; or (ii) the subject must have a genetic mutation PTCH; or (iii) the subject must have at least 6 BCC lesions and a genetic mutation PTCH.

In some embodiments, provided herein is a method of treatment of basal cell carcinoma (BCC), comprising topically administering a pharmaceutical composition of this invention, to a subject in need thereof, wherein the subject eligible for the treatment is selected according to any one of the following criteria (i) the subject must have at least 6 facial BCC lesions; or (ii) the subject must have a genetic mutation PTCH; or (iii) the subject must have at least 6 BCC lesions and a genetic mutation PTCH. In some embodiments, provided herein is a method of treatment of basal cell carcinoma (BCC), comprising topically administering a pharmaceutical composition of this invention, to a subject in need thereof, wherein the subject eligible for the treatment is selected according to any one of the following criteria (i) the subject must have at least 6 facial BCC lesions; or (ii) the subject must have a genetic mutation PTCH; or (iii) the subject must have at least 6 facial BCC lesions and a genetic mutation PTCH.

In some embodiments, provided herein is a method of treatment of basal cell carcinoma (BCC) lesions and/or preventing, delaying and/or inhibiting the formation of new BCC lesions, comprising topically administering a pharmaceutical composition of this invention, to a subject in need thereof, wherein the subject eligible for the treatment is selected according to any one of the following criteria (i) the subject must have at least 6 BCC lesions; or (ii) the subject must have a genetic mutation PTCH; or (iii) the subject must have at least 6 BCC lesions and a genetic mutation PTCH, and wherein said subject suffers from Gorlin syndrome. In another embodiment, the subject must have at least 6 facial lesions. In another embodiment, the subject must have a genetic mutation PTCH. In another embodiment, the subject must have at least 6 facial BCC lesions and a genetic mutation PTCH.

In some embodiments, provided herein is a method of treatment of basal cell carcinoma (BCC) lesions and/or preventing, delaying and/or inhibiting the formation of new BCC lesions, comprising topically administering a pharmaceutical composition of this invention, to a subject in need thereof, wherein the subject must have at least 6 BCC lesions and wherein said subject suffers from Gorlin syndrome.

In some embodiments, provided herein is a method of treatment of basal cell carcinoma (BCC) lesions and/or preventing, delaying and/or inhibiting the formation of new BCC lesions, comprising topically administering a pharmaceutical composition of this invention, to a subject in need thereof, wherein the subject must have at least 6 facial BCC lesions, and wherein said subject suffers from Gorlin syndrome.

In some embodiments, provided herein is a method of treatment of basal cell carcinoma (BCC) lesions and/or preventing, delaying and/or inhibiting the formation of new BCC lesions, comprising topically administering a pharmaceutical composition of this invention to a subject in need thereof, wherein the subject must have a genetic mutation PTCH and wherein said subject suffers from Gorlin syndrome.

In some embodiments, provided herein is a method of treatment of basal cell carcinoma (BCC) lesions and/or preventing, delaying and/or inhibiting the formation of new BCC lesions, comprising topically administering a pharmaceutical composition of this invention, to a subject in need thereof, wherein the subject must have at least 6 BCC lesions and a genetic mutation PTCH and wherein said subject suffers from Gorlin syndrome.

In some embodiments, provided herein is a method of treatment of basal cell carcinoma (BCC) lesions and/or preventing, delaying and/or inhibiting the formation of new BCC lesions, comprising topically administering a pharmaceutical composition of this invention, to a subject in need thereof, wherein the subject must have at least 6 facial BCC lesions and a genetic mutation PTCH and wherein said subject suffers from Gorlin syndrome.

In some embodiments, provided herein is a method of treatment of basal cell carcinoma (BCC) lesions and/or preventing, delaying and/or inhibiting the formation of new BCC lesions, comprising topically administering a pharmaceutical composition of this invention, to a subject in need thereof, wherein the subject must have at least 6 BCC lesions, and wherein said subject suffers from immunosuppression or said subject was exposed to radiation, asbestos, sun, or tanning salons. In another embodiment, the subject must have at least 8 BCC lesions. In another embodiment, the subject must have at least 10 BCC lesions. In another embodiment, the subject must have at least 12 BCC lesions. In another embodiment, the subject must have at least 6 facial BCC lesions. In another embodiment, the subject must have at least 8 facial BCC lesions. In another embodiment, the subject must have at least 10 facial BCC lesions. In another embodiment, the subject must have at least 12 facial BCC lesions.

In some embodiments, provided herein is a method of treatment of basal cell carcinoma (BCC) lesions and/or preventing, delaying and/or inhibiting the formation of new basal cell carcinoma lesions wherein the subject must have at least 6 facial BCC lesions, and wherein said subject suffers from immunosuppression or said subject was exposed to radiation, asbestos, sun, or tanning salons.

In some embodiments, provided herein is a method of treatment of basal cell carcinoma (BCC) lesions and/or preventing, delaying and/or inhibiting the formation of new BCC lesions, comprising topically administering a pharmaceutical composition of this invention, to a subject in need thereof, wherein the subject must have at least 6 BCC lesions, and wherein said subject suffers from a disorder comprising of non-melanoma skin cancer, Rombo syndrome, Bazex-Dupré-Christol syndrome, Xeroderma pigmentosum, Muir-Torre syndrome, Oculocutaneous albinism, or Gorlin syndrome.

In some embodiments, provided herein is a method of treatment of basal cell carcinoma (BCC) lesions and/or preventing, delaying and/or inhibiting the formation of new BCC lesions, comprising topically administering a pharmaceutical composition of this invention, to a subject in need thereof, wherein the subject must have at least 6 facial BCC lesions, and wherein said subject suffers from a disorder comprising of non-melanoma skin cancer, Rombo syndrome, Bazex-Dupré-Christol syndrome, Xeroderma pigmentosum, Muir-Torre syndrome, Oculocutaneous albinism, or Gorlin syndrome.

In some embodiments, provided herein is a method of treatment of basal cell carcinoma (BCC) lesions and/or preventing, delaying and/or inhibiting the formation of new BCC lesions, comprising topically administering a pharmaceutical composition of this invention, to a subject in need thereof, wherein the subject must have at least 6 BCC lesions, and wherein said subject suffers from Rombo syndrome, Bazex-Dupre-Christol syndrome, Xeroderma pigmentosum, Muir-Torre syndrome, or Oculocutaneous albinism.

In some embodiments, provided herein is a method of treatment of basal cell carcinoma (BCC) lesions and/or preventing, delaying and/or inhibiting the formation of new BCC lesions, comprising topically administering a pharmaceutical composition of this invention to a subject in need thereof, wherein the subject must have at least 6 facial BCC lesions, and wherein said subject suffers from Rombo syndrome, Bazex-Dupre-Christol syndrome, Xeroderma pigmentosum, Muir-Torre syndrome, or Oculocutaneous albinism.

In some embodiments, provided herein is a pharmaceutical composition of this invention for use in treating of basal cell carcinoma (BCC) in a subject in need thereof. In some embodiments, provided herein is a pharmaceutical composition of this invention for use in preventing, delaying and/or inhibiting the formation of new basal cell carcinoma in a subject in need thereof.

In some embodiments, provided herein is a pharmaceutical composition of this invention for use in treating BCC and/or preventing, delaying and/or inhibiting the formation of new basal cell carcinoma in a subject in need thereof, wherein the subject has at least one of (i) at least 1 BCC lesion; (ii) a genetic mutation PTCH; and/or (iii) at least one disorder comprising non-melanoma skin cancer, Rombo syndrome, Bazex-Dupre-Christol syndrome, Xeroderma pigmentosum, Muir-Torre syndrome, Oculocutaneous albinism, Gorlin syndrome, or any combination thereof.

In some embodiments, provided herein is a pharmaceutical composition of this invention for use in treating BCC and/or preventing, delaying and/or inhibiting the formation of new basal cell carcinoma in a subject in need thereof, wherein the subject has wherein the subject has at least one of (i) the subject must have at least 6 facial BCC lesions; or (ii) the subject must have a genetic mutation PTCH; or (iii) the subject must have at least 6 BCC lesions and a genetic mutation PTCH; and wherein said subject suffers from Gorlin syndrome.

In some embodiments, the method of this invention of treatment BCC resulting in a reduction of BCC lesion size or clinically resolved over time. In another embodiment, the method of this invention of treatment BCC resulting in a reduction of BCC lesion size. In another embodiment, the BCC lesion size is reduced by about 1-100%. In another embodiment, the lesion size is reduced by about 5%, 10%, 15%, 20%, 25%, 30%, 35%, 40%, 45%, 50%, 55%, 60%, 65%, 70%, 75%, 80%, 85%, 90%, 95%, or by 100%. In another embodiment, the lesion size is reduced by about 5%. In another embodiment, the BCC lesion size is reduced by about 10%. In another embodiment, the BCC lesion size is reduced by about 20%. In another embodiment, the lesion size is reduced by about 30%. In another embodiment, the lesion size is reduced by about 40%. In another embodiment, the lesion size is reduced by about 50%. In another embodiment, the BCC lesion size is reduced by about 60%. In another embodiment, the BCC lesion size is reduced by about 70%. In another embodiment, the lesion size is reduced by about 75%. In another embodiment, the BCC lesion size is reduced by about 80%. In another embodiment, the BCC lesion size is reduced by about 85%. In another embodiment, the BCC lesion size is reduced by about 90%. In another embodiment, the BCC lesion size is reduced by about 95%. In another embodiment, the BCC lesion size is reduced by about 100%. In another embodiment, the BCC lesion size is reduced by more than 5%, 10%, 15%, 20%, 25%, 30%, 35%, 40%, 45%, 50%, 55%, 60%, 65%, 70%, 75%, 80%, 85%, 90%, 95%, or 100%. In another embodiment, the BCC lesion size is reduced by between 1%-100%, 10%-100%, 20%-100%, 10%-20%, 10%-30%, 10%-40%, 10%-50%, 10%-60%, 10%-70%, 10%-80%, 10%-90%, 20%-30%, 20%-40%, 10%-20%. In another embodiment, the BCC lesion size is reduced by between 10%-30%. In another embodiment, the BCC lesion size is reduced by between 10%-30%.

In another embodiment, the treatment of BCC results in clinically resolved BCC, particularly the BCC disappeared completely. In another embodiment, the treatment of BCC lesion results in clinically resolved BCC, wherein the lesion is no longer suspected as BCC.

In some embodiments, the method of this invention prevents and/or inhibit and/or delays formation of new BCC, wherein the subject eligible for the prevention is selected according to any one of the following criteria (i) the subject must have at least 6 BCC lesions; or (ii) the subject must have a genetic mutation PTCH; or (iii) the subject must have at least 6 BCC lesions and a genetic mutation PTCH. In another embodiment, the subject must have at least 6 facial BCC lesions. In another embodiment, the subject must have at least 6 facial BCC lesions and a genetic mutation PTCH.

In some embodiments, the method of this invention prevents or inhibit formation of nSEBs, wherein the subject eligible for the prevention is selected according to any one of the following criteria (i) the subject must have at least 6 BCC lesions; or (ii) the subject must have a genetic mutation PTCH; or (iii) the subject must have at least 6 BCC lesions and a genetic mutation PTCH. In another embodiment, the subject must have at least 6 facial BCC lesions and a genetic mutation PTCH. In another embodiment, the subject must have at least 6 facial BCC lesions. In another embodiment, the subject must have at least 6 facial BCC lesions and a genetic mutation PTCH.

In some embodiments, the method of this invention reduces the number of nSEBs, wherein the subject is selected according to any one of the following criteria (i) the subject must have at least 6 BCC lesions; or (ii) the subject must have a genetic mutation PTCH; or (iii) the subject must have at least 6 BCC lesions and a genetic mutation PTCH. In another embodiment, the subject must have at least 6 facial BCC lesions. In another embodiment, the subject must have at least 6 facial BCC lesions and a genetic mutation PTCH.

In some embodiments, the present invention provides a method of treatment of basal cell carcinoma (BCC) lesions and/or preventing, delaying and/or inhibiting the formation of new BCC lesions, comprising topically administering the pharmaceutical composition of this invention, to a subject in need thereof, wherein the subject eligible for the treatment and/or prevention is selected according to any one of the following criteria (i) the subject must have at least 6 BCC lesions; or (ii) the subject must have a genetic mutation PTCH; or (iii) the subject must have at least 6 BCC lesions and a genetic mutation PTCH; wherein the composition is topically administered once daily, twice daily, trice daily, every other day, or three times a week.

In some embodiments, the present invention provides a method of treatment of basal cell carcinoma (BCC) lesions and/or preventing, delaying and/or inhibiting the formation of new BCC lesions, comprising topically administering a pharmaceutical composition of this invention, to a subject in need thereof, wherein the subject eligible for the treatment and/or prevention is selected according to any one of the following criteria (i) the subject must have at least 6 facial BCC lesions; or (ii) the subject must have a genetic mutation PTCH; or (iii) the subject must have at least 6 BCC lesions and a genetic mutation PTCH; wherein the composition is topically administered once daily, twice daily, trice daily, every other day, or three times a week. In another embodiment, the subject must have at least 6 facial BCC lesions and a genetic mutation PTCH.

In some embodiments, provided herein is a method of treatment of basal cell carcinoma (BCC) lesions and/or preventing, delaying and/or inhibiting the formation of new BCC lesions, once daily, twice daily, trice daily, every other day, or three times a week topical application of therapeutically effective amounts of the pharmaceutical composition of this invention.

In some embodiments, provided herein is a method of treatment of basal cell carcinoma (BCC) lesions and/or preventing, delaying and/or inhibiting the formation of new BCC lesions, comprising once daily, twice daily, trice daily, every other day, or three times a week topical application of therapeutically effective amounts of the pharmaceutical composition of this invention, to the facial skin of the subject in need thereof until said BCC is cured, prevented or alleviated or according to doctor's instructions, and wherein the subject eligible for the treatment and/or prevention is selected according to any one of the following criteria (i) the subject must have at least 6 facial BCC lesions; or (ii) the subject must have a genetic mutation PTCH; or (iii) the subject must have at least 6 BCC lesions and a genetic mutation PTCH. In another embodiment, the subject must have at least 6 facial BCC lesions and a genetic mutation PTCH.

In some embodiments, provided herein is a method of treatment of basal cell carcinoma (BCC) lesions and/or preventing, delaying and/or inhibiting the formation of new BCC lesions, comprising once daily, twice daily, trice daily, every other day, or three times a week topical application of therapeutically effective amounts of the pharmaceutical composition of this invention to affected skin area of the subject in need thereof until said BCC is cured, prevented or alleviated or according to doctor's instructions, and wherein the subject eligible for the treatment and/or prevention is selected according to one of the following criteria (i) the subject must have at least 6 BCC lesions; or (ii) the subject must have a genetic mutation PTCH; or (iii) the subject must have at least 6 BCC lesions and a genetic mutation PTCH. In another embodiment, the subject must have at least 6 facial BCC lesions. In another embodiment, the subject must have at least 6 facial BCC lesions and a genetic mutation PTCH.

In some embodiments, provided herein is a method of treatment of basal cell carcinoma (BCC) lesions and/or preventing, delaying and/or inhibiting the formation of new BCC lesions, comprising once daily, twice daily, trice daily, every other day, or three times a week topical application of therapeutically effective amounts of the pharmaceutical composition of this invention to affected skin area of the subject in need thereof until said BCC is cured, prevented or alleviated or according to doctor's instructions, and wherein the subject eligible for the treatment and/or prevention is selected according to one of the following criteria (i) the subject must have at least 6 facial BCC lesions; or (ii) the subject must have a genetic mutation PTCH; or (iii) the subject must have at least 6 BCC lesions and a genetic mutation PTCH. In another embodiment, the subject must have at least 6 facial BCC lesions and a genetic mutation PTCH.

In some embodiments, provided herein is a method of treatment of basal cell carcinoma (BCC) lesions and/or preventing, delaying and/or inhibiting the formation of new BCC lesions, comprising once daily, twice daily, trice daily, every other day, or three times a week topical application of therapeutically effective amounts of the pharmaceutical composition of this invention to any area of the body of the subject in need thereof until said BCC is cured, prevented or alleviated or according to doctor's instructions, and wherein the subject eligible for the treatment and/or prevention is selected according to one of the following criteria (i) the subject must have at least 6 BCC lesions; or (ii) the subject must have a genetic mutation PTCH; or (iii) the subject must have at least 6 BCC lesions and a genetic mutation PTCH. In another embodiment, the subject must have at least 6 facial BCC lesions. In another embodiment, the subject must have at least 6 facial BCC lesions and a genetic mutation PTCH.

In some embodiments, provided herein is a method of treatment of basal cell carcinoma (BCC) lesions and/or preventing, delaying and/or inhibiting the formation of new BCC lesions, comprising once daily, twice daily, trice daily, every other day, or three times a week topical application of therapeutically effective amounts of the pharmaceutical composition of this invention to the entire body of the subject in need thereof until said BCC is cured, prevented or alleviated or according to doctor's instructions, and wherein the subject eligible for the treatment and/or prevention is selected according to one of the following criteria (i) the subject must have at least 6 BCC lesions; or (ii) the subject must have a genetic mutation PTCH; or (iii) the subject must have at least 6 BCC lesions and a genetic mutation PTCH. In another embodiment, the subject must have at least 6 facial BCC lesions. In another embodiment, the subject must have at least 6 facial BCC lesions and a genetic mutation PTCH.

In some embodiments, provided herein is a method of treatment of BCC lesions comprising once daily, twice daily, trice daily, every other day, trice daily, every other day, or three times a week topical application of therapeutically effective amounts of the pharmaceutical composition of this invention on the BCC lesion of the subject in need thereof until said BCC is cured, prevented or alleviated or according to doctor's instructions, and wherein (i) the subject must have at least 6 BCC lesions; or (ii) the subject must have a genetic mutation PTCH; or (iii) the subject must have at least 6 BCC lesions and a genetic mutation PTCH.

In some embodiments, provided herein is a method of treatment of basal cell carcinoma (BCC) lesions and/or preventing, delaying and/or inhibiting the formation of new BCC lesions, lesions comprising once daily, twice daily, trice daily, every other day, trice daily, every other day, or three times a week topical application of therapeutically effective amounts of the pharmaceutical composition of this invention on the BCC lesion and on the skin surrounding the BCC lesion of the subject in need thereof until said BCC is cured, prevented or alleviated or according to doctor's instructions, and wherein (i) the subject must have at least 6 BCC lesions; or (ii) the subject must have a genetic mutation PTCH; or (iii) the subject must have at least 6 BCC lesions and a genetic mutation PTCH. In another embodiment, the subject must have at least 6 facial BCC lesions. In another embodiment, the subject must have at least 6 facial BCC lesions and a genetic mutation PTCH.

In some embodiments, provided herein is a method of treatment of BCC lesions comprising once daily, twice daily, trice daily, every other day, trice daily, every other day, or three times a week topical application of therapeutically effective amounts of the pharmaceutical composition of this invention on the BCC lesion of the subject in need thereof until said BCC is cured, prevented or alleviated or according to doctor's instructions, and wherein the subject has (i) at least 6 facial BCC lesions; or (ii) a genetic mutation PTCH; or (iii) at least 6 facial BCC lesions and a genetic mutation PTCH.

In some embodiments, provided herein is a method of treatment of basal cell carcinoma (BCC) lesions and/or preventing, delaying and/or inhibiting the formation of new BCC lesions, comprising topically administered the pharmaceutical composition of this invention, to a subject in need thereof, wherein the composition is topically administered for a period of about 3 months, about 6 months, about 9 months, about 12 months, about 18 months, about 24 months, about 36 months, chronically or for a lifetime; and wherein (i) the subject must have at least 6 BCC lesions; or (ii) the subject must have a genetic mutation PTCH; or (iii) the subject must have at least 6 BCC lesions and a genetic mutation PTCH. Each represents a separate embodiment of this invention. In another embodiment, the subject must have at least 6 facial BCC lesions. In another embodiment, the subject must have at least 6 facial BCC lesions and a genetic mutation PTCH.

In another embodiment, the composition is topically administered for a period of about 6 months. In another embodiment, the composition is topically administered for a period of about 9 months. In another embodiment, the composition is topically administered for a period of about 12 months. In another embodiment, the composition is topically administered for a period of more than 12 months. In another embodiment, the composition is topically administered for a period of a lifetime. In another embodiment, the composition is topically administered chronically.

In some embodiments, provided herein is a method of treatment of basal cell carcinoma (BCC) lesions and/or preventing, delaying and/or inhibiting the formation of new BCC lesions, comprising topically administering the pharmaceutical composition of this invention, to a subject in need thereof, wherein the composition is administered for a period of administration of more than 12 months, more than 18 months, more than 24 months, more than 36 months, at least 1-10 years, more than 1 year, more than 2 years, more than 3 years, more than 4 years, more than 5 years, more than 6 years, more than 7 years, more than 8 years, more than 9 years, more than 10 years, or chronically, or for a lifetime; and wherein the subject eligible for the treatment and/or prevention is selected according to one of the following criteria (i) the subject must have at least 6 BCC lesions; or (ii) the subject must have a genetic mutation PTCH; or (iii) the subject must have at least 6 BCC lesions and a genetic mutation PTCH. Each represents a separate embodiment of this invention. In another embodiment, the subject must have at least 6 facial BCC lesions. In another embodiment, the subject must have at least 6 facial BCC lesions and a genetic mutation PTCH. In another embodiment, the subject does not develop drug resistance. In another embodiment, the subject does not develop drug resistance following a discontinuation period of administration during the treatment.

In some embodiment, the subject of the methods provided in this invention does not develop drug resistance. In another embodiment, the subject of the methods provided in this invention does not develop drug resistance following a discontinuation period of administration during the treatment.

In some embodiments, provided herein is a method of treatment of basal cell carcinoma (BCC) lesions and/or preventing, delaying and/or inhibiting the formation of new BCC lesions, comprising topically administered the pharmaceutical composition of this invention, to a subject in need thereof, wherein the composition is topically administered for a period of more than 12 months; wherein the subject eligible for the treatment and/or prevention is selected according to one of the following criteria (i) the subject must have at least 6 BCC lesions; or (ii) the subject must have a genetic mutation PTCH; or (iii) the subject must have at least 6 BCC lesions and a genetic mutation PTCH; and wherein said subject does not develop drug resistance. Each represents a separate embodiment for this invention. In another embodiment, the composition is administered for a period of more than 18 months. In another embodiment, the composition is administered for a period of more than 24 months. In another embodiment, the composition is administered for a period of a lifetime. In another embodiment, the composition is administered chronically. In another embodiment, the subject must have at least 6 facial BCC lesions. In another embodiment, the subject must have at least 6 facial BCC lesions and a genetic mutation PTCH. In another embodiment, the subject does not develop drug resistance following a discontinuation period of administration during the treatment.

In some embodiments, provided herein a method of treatment of BCC lesions and/or preventing, delaying and/or inhibiting the formation of new BCC lesions, comprising topically administering the pharmaceutical composition of this invention, to a subject wherein the subject eligible for the treatment and/or prevention is selected according to any one of the following criteria (i) the subject must have at least 6 BCC lesions; or (ii) the subject must have a genetic mutation PTCH; or (iii) the subject must have at least 6 BCC lesions and a genetic mutation PTCH, wherein the subject does not develop drug resistance. In another embodiment, wherein the subject does not develop drug resistance following a discontinuation period of administration during the treatment. In some embodiments, the period of administration of the methods of this invention does not affect the drug efficacy. In another embodiment, following a discontinuation period of administration during the treatment, the drug efficacy is not affected.

In some embodiments, provided herein a method of treatment and/or prevention of basal cell carcinoma, comprising topically administering a pharmaceutical composition comprising patidegib or a pharmaceutically acceptable salt thereof, and a pharmaceutically acceptable carrier, to a subject wherein (i) the subject must have at least 6 facial BCC lesions; or (ii) the subject must have a genetic mutation PTCH; or (iii) the subject must have at least 6 BCC lesions and a genetic mutation PTCH, and wherein following a discontinuation period of administration during the treatment, the drug efficacy is not affected.

In some embodiments, the methods provided herein, the period of administration comprises at least consecutive 12 months of administration.

In some embodiments, the methods provided herein, the period of administration comprises (i) consecutive 12 months of administration (ii) followed by a discontinuation period (iii) followed by continuation of administration, wherein drug efficacy is not affected by the discontinuation period of administration during the treatment. In another embodiment, the discontinuation period is of 1, 2, 3, 4, 5, 6, 7, 8 months or between 1-3 months, 1-24 months, between 2-12 months, between 3-12 months, between 3-8 months, between 3-10 months.

In some embodiments, provided herein is a method of treatment of BCC lesions and/or preventing, delaying and/or inhibiting the formation of new BCC lesions, comprising topically administered the pharmaceutical composition of this invention, to a subject in need thereof, wherein the composition is topically administered for a period of at least 9 months, wherein after 9 months of administration the efficacy in treating and/or preventing of BCC is improved in comparison to the administration of the vehicle, and wherein (i) the subject must have at least 6 BCC lesions; or (ii) the subject must have a genetic mutation PTCH; or (iii) the subject must have at least 6 BCC lesions and a genetic mutation PTCH. In another embodiment, the subject must have at least 6 facial BCC lesions. In another embodiment, the subject must have at least 6 facial BCC lesions and a genetic mutation PTCH.

In some embodiments, provided herein is a method of treatment of clinically resolving SEB comprising topically administered the pharmaceutical composition of this invention, to a subject in need thereof, wherein the composition is topically administered for a period of at least 9 months, wherein after 9 months of administration the efficacy in treating and/or preventing of BCC is improved in comparison to the administration of the vehicle, and wherein the subject eligible for the treatment and/or prevention is selected according to one of the following criteria (i) the subject must have at least 6 BCC lesions; or (ii) the subject must have a genetic mutation PTCH; or (iii) the subject must have at least 6 BCC lesions and a genetic mutation PTCH. In another embodiment, the subject must have at least 6 facial BCC lesions. In another embodiment, the subject must have at least 6 facial BCC lesions and a genetic mutation PTCH.

In some embodiments, provided herein is a method of treatment of basal cell carcinoma (BCC) lesions and/or preventing, delaying and/or inhibiting the formation of new BCC lesions, comprising topically administered the pharmaceutical composition of this invention, to a subject in need thereof, wherein the composition is topically administered for a period of more than 12 months, wherein after 12 months of administration the efficacy in treating and/or preventing of BCC is improved in comparison to the administration of the vehicle, and wherein (i) the subject must have at least 6 BCC lesions; or (ii) the subject must have a genetic mutation PTCH; or (iii) the subject must have at least 6 BCC lesions and a genetic mutation PTCH. In another embodiment, the subject must have at least 6 facial BCC lesions. In another embodiment, the subject must have at least 6 facial BCC lesions and a genetic mutation PTCH.

In some embodiments, provided herein is a method of treatment of basal cell carcinoma (BCC) lesions and/or preventing, delaying and/or inhibiting the formation of new BCC lesions, comprising topically administered the pharmaceutical composition of this invention, to a subject in need thereof, wherein the composition is topically administered for a period of at least 12 months, wherein after 12 months of administration the efficacy in treating and/or preventing of BCC is not affected in comparison to the efficacy in treating/preventing in the first 12 months of topically administered the pharmaceutical composition of this invention, and wherein (i) the subject must have at least 6 BCC lesions; or (ii) the subject must have a genetic mutation PTCH; or (iii) the subject must have at least 6 BCC lesions and a genetic mutation PTCH. In another embodiment, the subject must have at least 6 facial BCC lesions. In another embodiment, the subject must have at least 6 facial BCC lesions and a genetic mutation PTCH.

In some embodiments, provided herein is a method of treatment of basal cell carcinoma (BCC) lesions and/or preventing, delaying and/or inhibiting the formation of new BCC lesions, comprising topically administered the pharmaceutical composition of this invention, to a subject in need thereof, wherein the composition is topically administered for a period of at least 18 months, wherein after 18 months of administration the efficacy in treating and/or preventing of BCC is not affected, and wherein (i) the subject must have at least 6 BCC lesions; or (ii) the subject must have a genetic mutation PTCH; or (iii) the subject must have at least 6 BCC lesions and a genetic mutation PTCH.

In another embodiment, the subject must have at least 6 facial BCC lesions. In another embodiment, the subject must have at least 6 facial BCC lesions and a genetic mutation PTCH.

In some embodiments, provided herein is a method of treatment of basal cell carcinoma (BCC) lesions and/or preventing, delaying and/or inhibiting the formation of new basal cell carcinoma lesions, to a subject in need thereof, wherein the composition is topically administered for a period of about 3 months, about 6 months, about 9 months, about 12 months, chronically, or for a lifetime; and wherein (i) the subject must have at least 6 facial BCC lesions; or (ii) the subject must have a genetic mutation PTCH; or (iii) the subject must have at least 6 BCC lesions and a genetic mutation PTCH.

In some embodiments, provided herein is a method of treatment of basal cell carcinoma (BCC) lesions and/or preventing, delaying and/or inhibiting the formation of new basal cell carcinoma lesions, to a subject in need thereof, wherein the number BCC surgeries is reduced; and wherein (i) the subject must have at least 6 BCC lesions; or (ii) the subject must have a genetic mutation PTCH; or (iii) the subject must have at least 6 BCC lesions and a genetic mutation PTCH.

In another embodiment, the BCC surgeries are reduced following 12 months of treatment. In another embodiment, the BCC lesions are facial BCC lesions.

In some embodiments, “substantially pure” refers to ≥97% of pure compound.

In some embodiments, “substantially free of impurity A” refers to patidegib compound with less than 0.05% wt % of impurity A. In some embodiments “substantially free of impurity A” refers to patidegib compound entirely absent/free of impurity A. In some embodiments, “substantially free of impurity B” refers to patidegib compound with less than 0.5 wt % of impurity B. In some embodiments “substantially free of impurity B” refers to patidegib compound entirely absent/free of impurity B.

In some embodiments “less than the detected limit” as used herein refers to less than <0.05 wt % of impurity A, and/or less than <0.015 wt % of impurity B.

In some embodiments “pharmaceutically acceptable salt” as used herein refers to a commonly used salts to form alkali metal salts of free acids and to form addition salts of free bases. The nature of the salt is not critical, provided that it is pharmaceutically acceptable. The term “pharmaceutically acceptable salts” also includes solvates of addition salts, such as hydrates, as well as polymorphs of addition salts. Suitable pharmaceutically acceptable acid addition salts can be prepared from an inorganic acid or from an organic acid. Examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid. In another embodiment, the salt is HCl salt.

In some embodiments “genetic mutation PTCH” refers to genetic mutation PTCH1 and/or genetic mutation PTCH2.

In some embodiments “treatment of BCC” refers to treating BCC including surgically eligible BCC until BCC has disappeared completely or reducing lesion growth (e.g. decrease of the size of a lesion/tumor). Moreover, “treatment of BCC” refers to treatment of BCC found on the entire body of the subject. In other embodiment, the term “treatment of BCC” refers to treating BCC including surgically eligible BCC until BCC is disappeared completely or reducing lesion growth (e.g. decrease of the size of a lesion/tumor) wherein the subject (i) must have at least 6 facial BCC lesions at baseline, before treatment; or (ii) the subject must have a genetic mutation PTCH; or (iii) the subject must have at least 6 BCC lesions at baseline, as baseline before treatment and a genetic mutation PTCH. In other embodiment, the term “treatment of BCC” refers to treating BCC including surgically eligible BCC until BCC is disappeared completely or reducing lesion growth (e.g. decrease of the size of a lesion/tumor) wherein the subject (i) must have at least 6 facial BCC lesions at baseline, before treatment; or (ii) the subject must have a genetic mutation PTCH; or (iii) the subject must have at least 6 facial BCC lesions at baseline, before treatment and a genetic mutation PTCH.

In some embodiments, “prevention of BCC” refers to preventing development of new BCC and new surgically eligible BCCs. Moreover, “prevention of BCC” refers to preventing development of new BCC and/or n-SEBs in the entire body.

In some embodiments, the term “delaying of new BCC refers to preventing development of new BCC and new surgically eligible BCCs. Moreover, “delaying of new BCC” refers to delaying development of new BCC and/or n-SEBs in the entire body. In another embodiment, “delaying of new BCC” refers to delaying the formation of new facial BCC.

In some embodiments, the term “inhibiting of new BCC refers to preventing development of new BCC and new surgically eligible BCCs. Moreover, “inhibiting of new BCC” refers to delaying development of new BCC and/or n-SEBs in the entire body. In another embodiment, “inhibiting of new BCC” refers to inhibiting the formation of new facial BCC.

In some embodiments, the term “Chronically” refers to more than 5 years, 10 years or longer, for lifetime.

In some embodiments, the term “drug resistance” refers to a drug that is no longer effective.

In some embodiments, the term “efficacy of the drug” refers to the effectiveness of the drug comprising prevention from reoccurrence of BCC and/or reduction in BCC lesion size/dimension over time.

In some embodiments, the treatment and/or prevention described herein refers to treatment of basal cell carcinoma (BCC) lesions and/or preventing, delaying and/or inhibiting the formation of new BCC lesions, wherein the subject must have at least 6 BCC lesions at baseline, before treatment in any of the entire body. “Entire body” refers to any specific part of the body (i.e. face, back, legs, hands, stomach etc., . . . ). Thus, the treatment comprises applying the pharmaceutical composition of this invention on the specific body part to be treated.

In some embodiments, the term “facial BCC lesions” refers to BCC lesions on the face.

In some embodiments, the term “BCC lesions” refers to BBC lesion in the entire body.

In some embodiments, the term “clinically resolved BCC” refers to no longer any visible evidence of a lesion consistent with BCC at the treated site a previously defined BBC lesion which is no longer BCC. In another embodiment, the term clinically resolved BCC refers to BCC which clinically disappeared completely.

In some embodiments, the term “nSEB” refers to BCCs with a longest diameter of ≥5 mm that:
a. were not surgically eligible BCCs (SEBs) at baseline;
b. have grown by ≥2 mm in longest diameter from baseline; and
c. have been verified histologically.

Moreover, nSEB refers to a histologically verified new BCC that should be surgically removed because of possible functional facial/health impairment as determined by the Investigator.

In some embodiments, the term “SEB” refers to surgically eligible BCCs with a longest diameter of ≥5 mm.

In some embodiments, the term “resolved SEB lesions” refers to BCCs lesions which no longer requires a surgical removal treatment. In another embodiment the term “resolved SEB lesions” refers to BCCs lesions with a diameter <5 mm or the BCC lesions have completely disappeared.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm”.

In some embodiments, “tertiary amine” as used herein refers to an amine in which the nitrogen atom is directly bonded to three carbons of any hybridization which cannot be carbonyl group carbons. The term “tertiary amine” as used herein refers to any tertiary amine known in the art. For example, but not limited to, trimethylamine, triethylamine, tripropylamine, triisopropylamine, tributylamine, tri-tert-butylamine, N,N-dimethylethanamine, N-ethyl-N-methylpropan-2-amine, N-ethyl-N-isopropylpropan-2-amine, diisopropylethylamine, N-methyl morpholine, pyridine and the like.

As used herein, numerical preceded by the term “%” or “wt %” which address to a compound (patidegib, impurity A or impurity B) refers to percentage by weight.

As used herein, numerical ranges preceded by the term “about” should not be considered to be limited to the recited range. Rather, numerical ranges preceded by the term “about” should be understood to include a range accepted by those skilled in the art for any given element in microcapsules or formulations according to the present invention.

In some embodiments, the term “about” as used herein means within an acceptable error range for a particular value as determined by one of ordinary skill in the art, which will depend in part on how the value is measured or determined, i.e., the limitations of the measurement system.

For example, “about” can mean a range of up to 10%, more preferably up to 5%, and still more preferably up to 1% of a given value. Where particular values are described in the application and claims, unless otherwise stated, the meaning of the term “about” is within an acceptable error range for the particular value.

The terms “comprise”, “comprising”, “includes”, “including”, “having” and their conjugates mean “including but not limited to”.

The term “consisting of” means “including and limited to”.

As used herein, the singular form “a”, “an” and “the” include plural references unless the context clearly dictates otherwise. For example, the term “a compound” or “at least one compound” may include a plurality of compounds, including mixtures thereof.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment.

Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

### Example 1

Preparation of Patidegib of this Invention

General chemistry methods. ¹H-NMR, ¹³C-NMR spectra were obtained on JEOL 400 MHz apparatus. ¹H chemical shifts are reported in comparison to solvent peaks from CDCl₃ (δH 7.26 ppm) or DMSO-d₆ (2.50 ppm). ¹³C chemical shifts are reported in comparison to solvent peaks from CDCl₃ (δC 77.00 ppm) or DMSO-d₆ (δC 39.50 ppm). In the ¹³C spectra a peak at 13.38 ppm is often encountered which is not related to the product.

LC/MS analysis was performed on Waters Acquity UPLC-H-class coupled to Xevo G2QTof for mass analysis. HPLC separation of the peaks was realized on Waters X-bridge C18 column (50×4.6 mm ID, 2.5 micron particle size) with mobile phase A (0.1% v/v TFA in H₂O) and B (0.1% v/v TFA in CH₃CN). The eluted product peaks were identified by mass spectrometry as their molecular ion via positive electron spray ionization (ES+). Mass spectra were analyzed with MassLynx v4.1.

### Preparation of Int 2

Compound 2 was synthesized as shown in U.S. Pat. No. 9,879,025. Compound 2 was mesylated with MsCl in the presence of triethylamine in 2-methyltetrahydrofuran (2-Me-THF), to obtain Int 2, as shown in scheme 2 and FIG. 1.

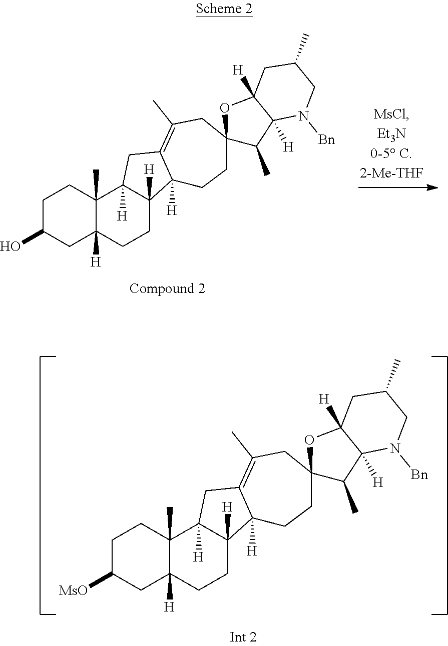

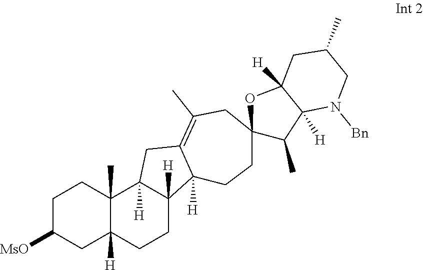

Chemical name of Int 2: (2S,3R,3aS,3′S,4a′R,6S,6a′R,6b'S,7aR,12a'S,12b'S)-4-benzyl-3,6,11′,12b′-tetramethyl-2′,3a,3′,4,4′,4a′,5,5′,6,6′,6a′,6b′,7,7a,7′,8′,10′,12′,12a′,12b′-icosahydro-1′H,3H-spiro[furo[3,2-b]pyridine-2,9′-naphtho[2,1-a]azulen]-3′-yl methanesulfonate.

### NMR Spectroscopy

¹H NMR spectrum (400 MHz, DMSO-d₆) ppm δ 7.36-7.24 (m), 4.92 (m), 3.93 (d), 3.85-3.72 (m), 3.58-3.52 (m), 3.49 (m), 3.14 (s), 2.87 (d), 2.60 (m), 2.43 (d), 2.15 (m), 2.05 (m), 1.98-1.88 (m), 1.82-1.60 (in), 1.62 (s), 1.60-1.14 (m), 1.12 (d), 0.98 (d), 0.91 (s), 0.87 (m), 0.78 (d).

¹³C NMR spectrum (100 MHz, DMSO-d₆) ppm δ 139.72, 138.62, 128.42, 128.18, 126.84, 123.83, 81.33, 79.72, 75.02, 74.27, 69.35, 66.71, 60.90, 58.93, 49.30, 48.85, 46.55, 43.26, 40.67, 39.76, 37.78, 37.21, 35.72, 34.01, 32.67, 31.07, 31.01, 30.40, 30.00, 28.75, 27.62, 27.08, 25.61, 25.41, 25.21, 23.70, 22.35, 20.88, 18.95, 9.86.

Mass spectrometry (ES+): The observed product peak [M+H]⁺=596.4 is conform with the structure (C₃₆H₅₄NO₄S⁺).

### Preparation of Int 3

Int 2 was converted to azide—Int 3 by using tetrabutylphosphonium azide (TBPA) in 2-Me-THF, as shown in scheme 3 and FIG. 1:

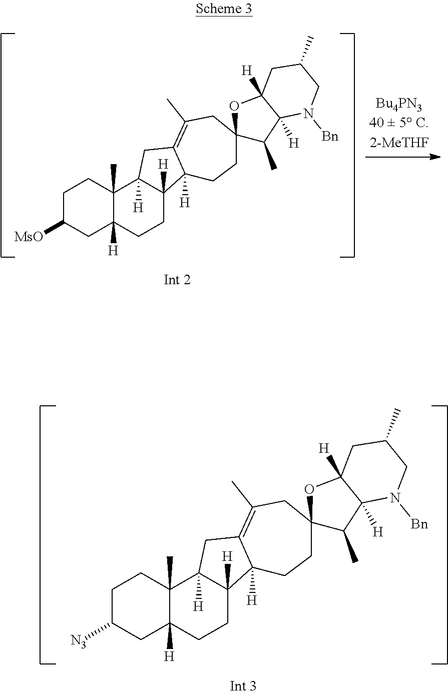

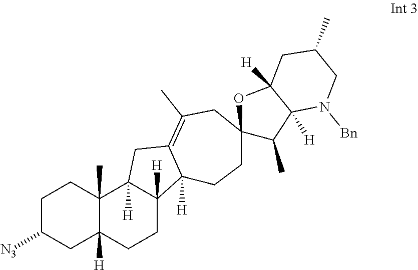

Chemical name: (2S,3R,3aS,3′R,4a′R,6S,6a′R,6b'S,7aR,12a'S,12b'S)-3′-azido-4-benzyl-3,6,11′,12b′-tetramethyl-2′,3a,3′,4,4′,4a′,5,5′,6,6′,6a′,6b′,7,7a,7′,8′,10′,12′, 12a′,12b′-icosahydro-1′H,3H-spiro[furo[3,2-b]pyridine-2,9′-naphtho[2,1-a]azulene].

### NMR Spectroscopy

¹H NMR spectrum (400 MHz, CDCl₃) ppm δ 7.35-7.31 (m), 7.25 (m), 3.99 (d), 3.91 (m), 3.71 (m), 3.63 (td), 3.26 (m), 2.92 (d), 2.75 (dd), 2.47 (d), 2.23 (m), 2.16-1.72 (m), 1.71 (s), 1.66-1.26 (m), 1.23 (d), 1.21-1.26 (m), 1.05 (d), 0.95-0.92 (m), 0.88 (s), 0.81 (d). Peaks at 3.91, 3.71 and 1.17 ppm correspond to the presence of residual 2-MeTHF. ¹³C NMR spectrum (100 MHz, CDCl₃) ppm δ 140.55, 138.72, 128.76, 128.22, 126.91, 124.48, 82.23, 75.82, 69.79, 61.41, 60.99, 59.71, 49.76, 49.45, 46.87, 43.83, 41.81, 41.62, 39.36, 37.69, 36.69, 34.46, 32.57, 30.10, 29.26, 28.24, 27.92, 26.94, 25.97, 23.77, 22.38, 19.11, 10.06.

Mass spectrometry (ES+). The observed product peak [M+H]⁺=543.4 is conform with the structure (C₃₅H₅₁N₄O⁺).

### Preparation of Int 4

The azide Int 3 was reduced by catalytic hydrogenation in the presence of platinum on carbon in 2-Me-THF as shown in scheme 4 and FIG. 1.

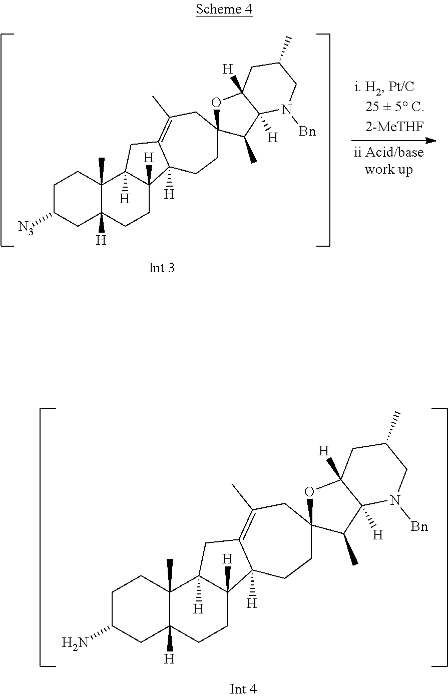

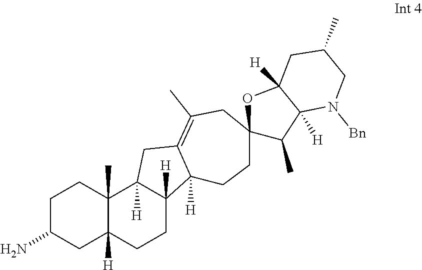

Chemical name: (2S,3R,3aS,3′R,4a′R,6S,6a′R,6b'S,7aR,12a'S,12b'S)-4-benzyl-3,6,11′,12b′-tetramethyl-2′,3a,3′,4,4′,4a′,5,5′,6,6′,6a′,6b′,7,7a,7′,8′,10′,12′,12a′,12b′-icosahydro-1′H,3H-spiro[furo[3,2-b]pyridine-2,9′-naphtho[2,1-a]azulen]-3′-amine

### NMR Spectroscopy

¹H NMR spectrum (400 MHz, CDCl₃) ppm δ 7.33-7.31 (m), 7.26 (m), 3.99 (d), 3.63 (td), 2.91 (d), 2.73 (dd), 2.64 (m), 2.45 (d), 2.23 (t), 2.17-1.74 (m), 1.70 (s), 1.68-1.12 (m), 1.07 (d), 0.96-0.90 (m), 0.87 (s), 0.82 (d). Peaks at 3.90 and 3.70 ppm correspond to the presence of residual 2-MeTHF.

¹³C NMR spectrum (100 MHz, CDCl₃) ppm δ 140.96, 138.69, 128.74, 128.20, 126.89, 124.10, 82.30, 75.78, 69.77, 61.39, 59.69, 51.25, 49.74, 49.54, 46.93, 43.77, 41.84, 41.82, 39.35, 37.82, 37.65, 37.29, 34.39, 31.84, 30.14, 29.23, 28.38, 27.88, 26.06, 23.71, 22.52, 19.10, 10.06.

Mass spectrometry (ES+): The observed product peak [M+H]+=517.4 is conform with the structure (C₃₅H₅₃N₂O⁺).

### Preparation of Compound 3

The amine Int 4, was sulfonylated under Schotten-Baumann type conditions with methanesulfonyl chloride (MsCl) and tribasic sodium phosphate (Na₃PO₄) is a biphasic mixture of water and 2-Me-THF, as shown on scheme 5 and FIG. 1—the yield of the synthetic steps from Compound 2 to Compound 3 (via int 2, int 3 and int 4) ranges between 71% to 83%.

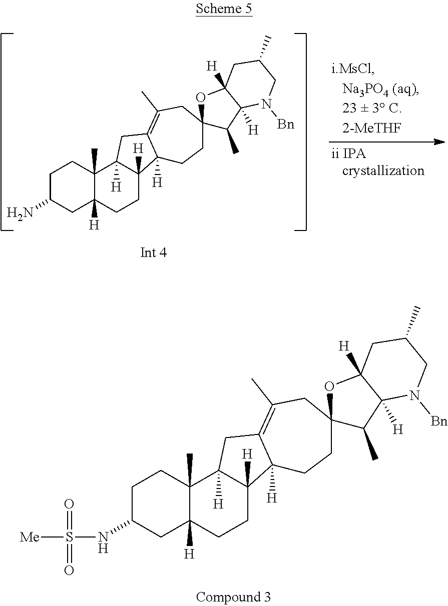

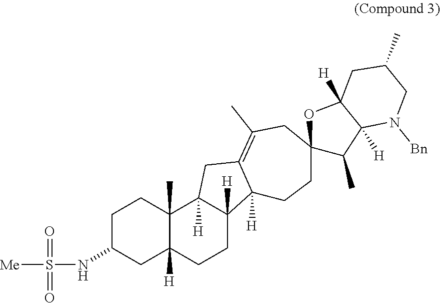

Chemical name: N-((2S,3R,3aS,3′R,4a′R,6S,6a′R,6b'S,7aR,12a'S,12b'S)-4-benzyl-3,6,11′,12b′-tetramethyl-2′,3a,3′,4,4′,4a′,5,5′,6,6′,6a′,6b′,7,7a,7′,8′,10′,12′,12a′,12b′-icosahydro-1′H,3H-spiro[furo[3,2-b]pyridine-2,9′-naphtho[2,1-a]azulen]-3′-yl)methanesulfonamide.

### NMR Spectroscopy

¹H NMR spectrum (400 MHz, CDCl₃) ppm δ 7.36-7.30 (m), 7.25 (m), 4.34 (d), 4.01 (septet), 3.99 (d), 3.63 (td), 3.29 (m), 2.96 (s), 2.91 (d), 2.74 (dd), 2.46 (d), 2.23 (dd), 2.16-1.72 (m), 1.70 (s), 1.67-1.25 (m), 1.20 (d), 1.15 (m), 1.07 (d), 0.94 (m), 0.88 (s), 0.82 (d). Residual iPrOH was observed in the ¹H spectrum at 4.01 ppm and 1.20 ppm.

¹³C NMR spectrum (100 MHz, CDCl₃) ppm δ 140.56, 138.66, 128.75, 128.22, 126.92, 124.50, 82.18, 75.78, 69.74, 64.32, 61.36, 59.68, 53.97, 49.73, 49.46, 46.83, 43.81, 42.12, 41.84, 41.79, 39.32, 37.68, 37.00, 35.18, 34.20, 30.08, 29.47, 29.21, 28.09, 27.89, 25.95, 25.31, 23.76, 22.37, 19.08, 10.05.

Mass spectrometry (ES+): The observed product peak [M+H]+=595.4 is conform with the structure (C₃₆H₅₅N₂O₃S⁺).

Preparation of Patidegib of this Invention

The benzyl group of Compound 3 was removed under catalytic hydrolysis conditions. The resulting free base Patidegib was reacted with hydrochloric acid to give the Patidegib of this invention, as shown in scheme 6 and FIG. 1—yield of this step ranges between 73% to 81%.

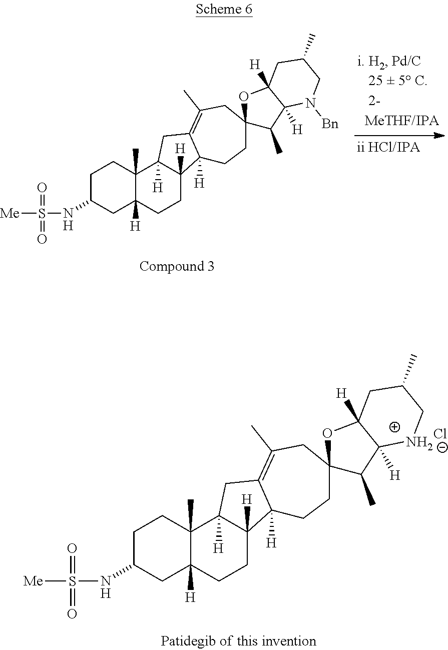

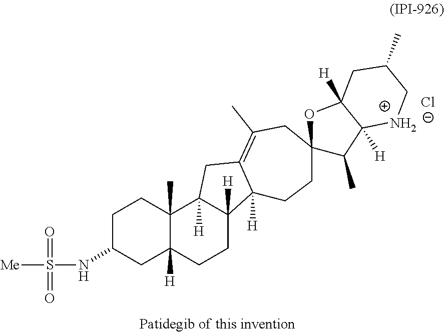

Chemical name: (2S,3R,3aS,3′R,4a′R,6S,6a′R,6b'S,7aR,12a'S,12b'S)-3,6,11′,12b′-tetramethyl-3′-(methylsulfonamido)-2′,3a,3′,4,4′,4a′,5,5′,6,6′,6a′,6b′,7,7a,7′,8′,10′, 12′,12a′,12b′-icosahydro-1′H,3H-spiro[furo[3,2-b]pyridine-2,9′-naphtho[2,1-a]azulen]-4-ium chloride, isopropanolate.

### NMR Spectroscopy

¹H NMR spectrum (400 MHz, DMSO-d₆) ppm δ 6.88 (d), 4.38 (d), 3.76 (m), 3.63 (td), 3.14 (m), 3.07 (m), 2.87 (s), 2.66 (t), 2.45 (d), 2.14 (m), 2.09-2.05 (m), 1.99 (m), 1.86-1.63 (m), 1.60 (s), 1.59-1.12 (m), 1.04 (d), 0.98 (d), 0.94 (d), 0.87 (s).

¹³C NMR spectrum (100 MHz, DMSO-d₆) ppm δ 139.98, 123.94, 82.73, 72.35, 62.00, 60.53, 52.79, 49.88, 48.95, 48.81, 46.57, 42.31, 41.23, 37.60, 36.75, 35.92, 34.28, 33.67, 29.92, 28.72, 27.73, 27.15, 26.84, 25.53, 25.47, 23.73, 22.05, 17.75, 10.44.

Mass spectrometry (ES+). The observed product peak [M+H]+=505.3 is conform with the structure (C₂₉H₄₉N₂O₃S⁺).

### Example 2

Comparison of Impurity Profile of Patidegib Manufactured by the Process Provided in U.S. Pat. No. 8,785,635 (US '635) and the Process Provided (Table 1):

TABLE 1

| | | | Maximum Level | |
|---|---|---|---|---|
| | | | Observed in GMP Lots | |
| | | | % of | |
| | | | impurity of | % of |
| | | | Patidegib | impurity of |
| | | | prepared | Patidegib |
| | | | according | prepared |
| | | | to the | according |
| | | | process of | to the |
| | | | U.S. Pat. No. | process |
| | | | 8,785,635 | provided |
| Impurity | Structure | How It Forms | (US ′635) | herein |
| Impurity A | 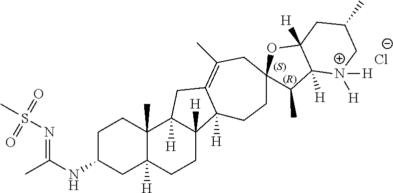 | Mesylated amidine analogue at 3′-NH₂ of IPI-926 (only in the process cited in US ′635). | 0.09 wt % | Not observed (N/A) |
| Impurity B | 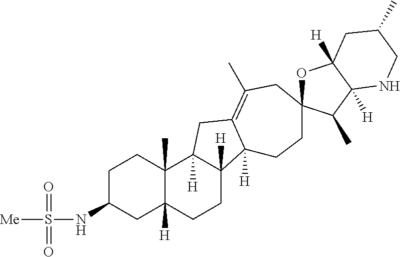 | 3′ epimer of Patidegib | 1.14 wt % | 0.14 wt % |

Impurity A was not observed in the batches of the patidegib obtained by the process provided herein. The highest-level impurity observed in the patidegib batches obtained by the process of U.S. Pat. No. 8,785,635 (US '635) is impurity B (C3-diastereomer of patidegib). This impurity is typically not detected in batches of the patidegib obtained by the process provided herein; or as presented in Table 1 lower concentration compared to US '635 (0.14 wt % in the process of this invention vs 1.14 wt % in the process of US '635).

1. A compound of patidegib or any pharmaceutically acceptable salt thereof, wherein the compound comprises less than 0.05 wt % of impurity A 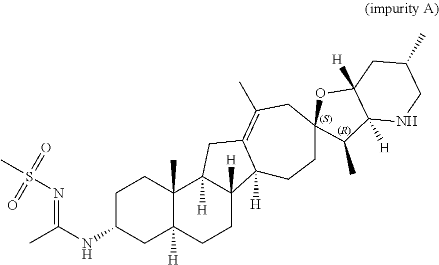
or any salt thereof, or less than 0.5 wt % of impurity B 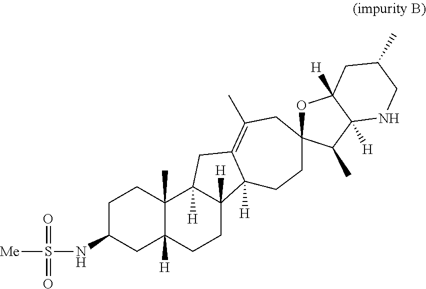
or any salt thereof, or any combination thereof.

2. The compound of claim 1, wherein the compound is at least 98% pure by weight.

3. The compound of claim 2, wherein the compound is at least 99% pure by weight.

4. The compound of claim 1, wherein the impurities comprise less than 0.05 wt % of impurity A or any salt thereof.

5. The compound of claim 1, wherein the impurities comprise less than 0.5 wt % of impurity B or any salt thereof.

6. The compound of claim 1, wherein the impurities comprise less than 0.4 wt % of impurity B or any salt thereof.

7. The compound of claim 1, wherein the impurities comprise less than 0.3 wt % of impurity B or any salt thereof.

8. The compound of claim 1, wherein the impurities comprise less than 0.2 wt % of impurity B or any salt thereof.

9. The compound of claim 1, wherein the impurities comprise less than 0.05 wt % of impurity A or any salt thereof and less than 0.5 wt % of impurity B or any salt thereof.

10. The compound of claim 1, wherein the impurities comprise less than 0.5 wt % of impurity B or any salt thereof and does not comprise impurity A.

11. A process for the preparation of the compound of patidegib of claim 1, wherein the process comprises the following steps:
a) preparing Compound 3 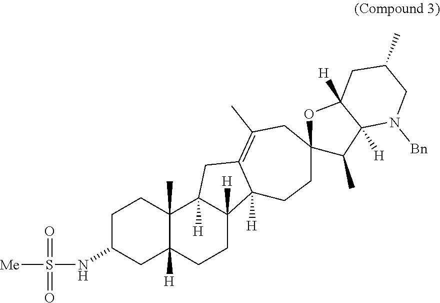
from Compound 2 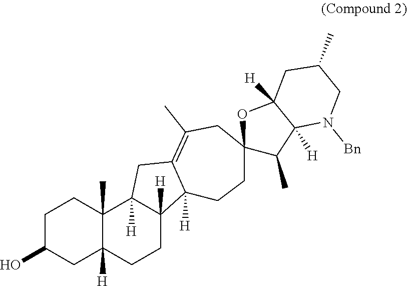
wherein the alcohol group of Compound 2 undergoes mesylation, followed by substitution of the mesylate group to an azide group, followed by reduction of the azide group into an amine group, and followed by mesylation of the amine group to obtain Compound 3;
b) deprotecting of the benzyl group of Compound 3; and
c) adding acid to obtain the salt of the compound of any one of claims 1-10.

12. The process of claim 11, wherein Compound 3 is further crystallized.

13. The process of claim 11, wherein the substitution of the mesylate group into an azide group comprises tetra-n-butylphosphonium azide.

14. The process of claim 11, wherein the reduction of the azide group into an amine group comprises catalytic hydrogenation followed by acid/base extractions.

15. The process of claim 14, wherein the catalytic hydrogenation of the azide into an amine comprises Pt/C or Ni/C catalyst with H₂.

16. The process of claim 14, wherein the acid/base extractions comprise HCl, 2-MeTHF and NaOH.

17. The process of claim 11, wherein the mesylation of the amine group comprises methanesulfonyl chloride (MsCl) and a base.

18. The process of claim 11, wherein the deprotecting of the benzyl group comprises catalytic hydrogenation.

19. The process of claim 18, wherein the catalytic hydrogenation comprises Pd/C H₂.

20. A compound of patidegib or any acceptable salt thereof, comprising less than 0.05 wt % of impurity A 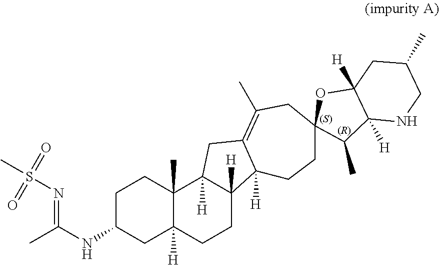
or any salt thereof, less than 0.5 wt % of impurity B 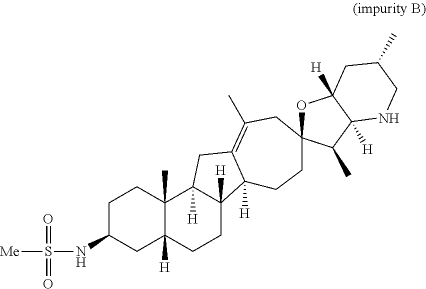
or any salt thereof, or any combination thereof prepared according to the process of claim 11.

21. A pharmaceutical composition comprising the compound of claim 1 and a pharmaceutically acceptable carrier.

22. A pharmaceutical composition comprising the compound of claim 20 and a pharmaceutically acceptable carrier.

23. The pharmaceutical composition of claim 21, wherein the pharmaceutical composition is formulated as a gel.

24. The pharmaceutical composition of claim 22, wherein the pharmaceutical composition is formulated as a gel.

25. A method of treatment of basal cell carcinoma (BCC), comprising topically administering, to a subject in need, the pharmaceutical composition according to claim 21.

26. A method of preventing, delaying and/or inhibiting the formation of new basal cell carcinoma in a subject, comprising topically administering to a subject a pharmaceutical composition according to claim 21.

27. The method of claim 25, wherein the subject has at least one of (i) at least 1 BCC lesion; (ii) a genetic mutation PTCH; and/or (iii) at least one disorder comprising non-melanoma skin cancer, Rombo syndrome, Bazex-Dupré-Christol syndrome, Xeroderma pigmentosum, Muir-Torre syndrome, Oculocutaneous albinism, Gorlin syndrome, or any combination thereof.

28. The method of claim 2, wherein the subject has at least one of (i) the subject must have at least 6 facial BCC lesions; or (ii) the subject must have a genetic mutation PTCH; or (iii) the subject must have at least 6 BCC lesions and a genetic mutation PTCH; and wherein said subject suffers from Gorlin syndrome.

29. The method of claim 25, wherein the subject has Gorlin Syndrome and a genetic mutation of PTCH1.

30. The method of claim 25, wherein the method of treatment basal cell carcinoma results in a reduction of BBC lesion size or clinically resolved BBC lesion over time.

31. The method of claim 26, wherein the method of prevention basal cell carcinoma lesions refers to preventing formation of new BCCs, or new surgically eligible BCCs.

32. The method of claim 25, wherein the pharmaceutical composition is topically administered once daily, twice daily, trice daily, every other day, or three times a week.

33. The method of claim 25, wherein the subject does not develop drug resistance.

34. The method of claim 25, wherein the patidegib is in an amount of about 0.1 wt % to about 6 wt % of the composition.

35. The method of claim 26, wherein the subject has at least one of (i) at least 1 BCC lesion; (ii) a genetic mutation PTCH; and/or (iii) at least one disorder comprising non-melanoma skin cancer, Rombo syndrome, Bazex-Dupre-Christol syndrome, Xeroderma pigmentosum, Muir-Torre syndrome, Oculocutaneous albinism, Gorlin syndrome, or any combination thereof.

36. The method of claim 26, wherein the subject has at least one of (i) the subject must have at least 6 facial BCC lesions; or (ii) the subject must have a genetic mutation PTCH; or (iii) the subject must have at least 6 BCC lesions and a genetic mutation PTCH; and wherein said subject suffers from Gorlin syndrome.

37. The method of claim 26, wherein the pharmaceutical composition is topically administered once daily, twice daily, trice daily, every other day, or three times a week.

38. The method of claim 26, wherein the subject does not develop drug resistance.

39. The method of claim 26, wherein the patidegib is in an amount of about 0.1 wt % to about 6 wt % of the composition.
